SEQUENCE LISTING (2 sequences)

SEQ ID NO: 1 - protein, 191 aa, Homo sapiens
MRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQ

SEQ ID NO: 2 - protein, 254 aa, Homo sapiens
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

(19) United States
(12) Patent Application Publication
(10) Pub. No.: US 2008/0248510 A1
(43) Pub. Date: Oct. 9, 2008

(54) HUMAN Fc GAMMA RECEPTOR III

(76) Inventors: Ulrich Brinkmann, Weilheim (DE); Peter Bruenker, Hittnau (CH); Claudia Ferrara Koller, Zuerich (CH); Martin Lanzendoerfer, Tutzing (DE); Joerg Thomas Regula, Muenchen (DE); Tilman Schlothauer, Penzberg (DE); Stefan Seeber, Penzberg (DE); Anne Zeck, Muenchen (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/056,310

(22) Filed: Mar. 27, 2008

(30) Foreign Application Priority Data
Apr. 3, 2007 (EP) 07006952.1
Jun. 4, 2007 (EP) 07010939.2

(51) Int. Cl. G01N 33/53; G01N 33/566; C12P 21/04; C07K 14/00

(52) U.S. Cl. 435/7.94; 436/501; 435/7.1; 435/7.92; 435/7.9; 435/69.1; 530/395

(57) ABSTRACT

The present invention relates to the field of human immunoglobulin receptors, specifically the glycostructure of a human Fc gamma receptor IIIa recombinantly expressed in human embryonic kidney cells and Chinese hamster ovary cells.

### PRIORITY TO RELATED APPLICATION(S)

This application claims the benefit of European Patent Application No. 07006952.1 filed Apr. 3, 2007, and European Patent Application 07010939.2, filed Jun. 4, 2007, which are hereby incorporated by reference in its entirety.

### FIELD OF THE INVENTION

The present invention relates to the field of human immunoglobulin receptors, specifically, a human Fc gamma receptor IIIa, as recombinantly expressed in human embryonic kidney cells and Chinese hamster ovary cells, and the glycostructure thereof.

### BACKGROUND OF THE INVENTION

An immunoglobulin comprises in general two light polypeptide chains and two heavy polypeptide chains. Each of the heavy and light polypeptide chains comprises a variable region (generally the amino terminal portion of the polypeptide chain) which contains one or more binding domains that are able to interact specifically with an antigen. Each of the heavy and light polypeptide chains also comprise a constant region (generally the carboxyl terminal portion). The constant region of the heavy chain mediates the binding of the immunoglobulin e.g. to cells bearing an Fc gamma receptor (FcγR), such as phagocytic cells, or to cells bearing the neonatal Fc receptor (FcRn) also known as Brambell receptor. It also mediates the binding to some factors including factors of the classical complement system such as component (C1q).

Immunoglobulin molecules are assigned to five different classes: IgA (immunoglobulin of class A), IgD, IgE, IgG and IgM. Within these classes the immunoglobulins differ in their over-all structure but the building blocks are quite similar.

Hulett and Hogarth (Hulett, M. D. and Hogarth, P. M., Adv. Immunol. 57 (1994) 1-127) reported that the extracellular receptors for the Fc part of immunoglobulins of class G are a family of transmembrane glycoproteins comprising three different receptor types having different binding specifity: FcγRI, FcγRII, and FcγRIII. Receptors of type I interact with uncomplexed IgG, whereas receptors of type II and III interact preferably with complexed IgG. Human FcγRIII (CD 16) exists in two isoforms and two polymorphic forms. The first isoform FcγRIIIa is a transmembrane molecule encoded by a different gene than the second isoform FcγRIIIb, which is a GPI-anchored membrane protein. The first polymorphic form V159 has a valine residue at position 159 of the amino acid sequences whereas the second polymorphic form F159 has a phenylalanine residue at position 159.

Takahashi et al. (Takahashi, N., et al., Glycobiology 12 (2002) 507-515) report the N-glycosylation profile of recombinant human soluble FcγRIII produced in BHK cells. The affinity of the interaction between FcγRIIIb ectodomains and monomeric human IgG subclasses was reported by Galon et al. (Galon, J., et al., Eur. J. Immunol. 27 (1997) 1928-1932). Ligand binding and phagocytosis by CD16 isoforms was reported by Nagarajan et al. (Nagarajan, S., et al., J. Biol. Chem. 270 (1995) 25762-25770). EP-A-1 314 741 reports bispecific anti-CD19×anti-CD16 antibodies and uses thereof.

It is an object of the current invention to provide the defined glycostructure, i.e. to provide a list of the oligosaccharides attached to specific positions, of human Fc gamma receptor IIIa.

### SUMMARY OF THE INVENTION

The current invention comprises several aspects in the field of the glycostructure of Fc gamma receptors. The first aspect is a recombinant human Fc gamma receptor IIIa, with the amino acid sequence of SEQ ID NO: 1, and comprising at amino acid position 163 of SEQ ID NO: 1 one of the following N-linked oligosaccharides:

### HexNAc(β1→4) [Fuc(α1→3(6))]GlcNAc(β1→2)Man(α1→6(3)) [HexNAc(β1→4)GlcNAc(β1→2)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3)) [Man(α1→6)Man(α1→3(6))]Man(β1→4)GlcNAc(β1→4) [Fuc(α1→6)]GlcNAcβ1-, or

### HexNAc(β1→4) [Fuc(α1→3(6))]GlcNAc(β1→2)Man(α1→6(3)) [HexNAc(β1→4) [Fuc(α1→3(6))]GcNAc(β1→2)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3)) [Man(α1→6)Man(α1→6)Man (α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3))[Man(α1→6(3))Man(α1→3(6))]Man(β1→6)GlcNAc(β1→4) [Fuc(α1→6)]GlcNAcβ1-, +HexNAc, or

### HexNAc(β1→4)[Fuc(α1→3(6))]GlcNAc(β1→2)Man(α1→6(3))[NeuAc(α2→6(3))Gal(β1→4)GlcNAc(β1→2)Man(α1→3(6))]Man(β1→4)GlcNAc(β1→4)[Fuc(α1→6)]GlcNAcβ1-, or

### Gal(β1→4)[Fuc(α1→3(6))]GlcNAc(β1→2)Man(α1→6)[Gal(α1→4) [Fuc(α1→3(6))]GlcNAc(β1→2)Man(α1→3)]Man(β1→4)GlcNAc(β1→4)[Fuc(α1→6)]GlcNAcβ1-, or

### Gal(β1→4) [Fuc(α1→3(6))]GlcNAc(β1→2)Man(α1→6(3))[Fuc(α1→3(6))GlcNAc(β1→2)Man(α1→3(6))]Man(β1→4)GlcNAc(β1→4)[Fuc(α1→6)]GlcNAcβ1-, or

### HexNAc(β1→4)GlcNAc(β1→2)Man (β1→4)GlcNAc(β1→4) [Fuc(α1→6)]GlcNacβ1-.

A second aspect of the invention is a recombinant human Fc gamma receptor IIIa, with the amino acid sequence of SEQ ID NO: 1, and comprising at amino acid position 163 of SEQ ID NO: 1 one of the following N-linked oligosaccharides:

### NeuAc(α2→6(3)Gal(β1→4)GlcNAc(β1→6)Man(α1→6) [Gal(β1→4)GlcNAc(β1→4)Man(α1→3)]Man(β1→4)GlcNAc(β1→4)[Fuc(α1→6)]GlcNAcβ1-, or

### NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4) [Fuc(α1→6)]GlcNAcβ1-.

A third aspect of the invention is a method for the determination of the binding of an immunoglobulin to a recombinant Fc gamma receptor according to the invention, comprising the following steps

i) providing an immunoglobulin to be analyzed,
ii) providing a recombinant Fc gamma receptor according to the invention,
iii) contacting the immunoglobulin with the Fc gamma receptor, and
iv) determining the binding of the immunoglobulin to the Fc gamma receptor.

Another aspect of the current invention is a composition of recombinant human Fc gamma receptor IIIa, whereby

a) the recombinant human Fc gamma receptor IIIa has the amino acid sequence of SEQ ID NO: 1 and has been expressed in HEK 293 cells,
b) the composition comprises a mixture of at least two recombinant human Fc gamma receptor IIIa of SEQ ID NO: 1 differing in the N-linked oligosaccharide at amino acid position 163 of SEQ ID NO: 1,
whereby the at least two different N-linked oligosaccharides are selected from:
none,

### HexNAc(β1→4) [Fuc(α1→3(6))]GlcNAc(β1→2)Man(α1→6(3))[HexNAc(β1→4)GlcNAc(β1→2)Man(α1→3(6))]Man(β1→4)GlcNAc(β1→4) [Fuc(α1→6)]GlcNAcβ1-,

### HexNAc(β1→4) [Fuc(α1→3(6))]GlcNAc(β1→2)Man(α1→6(3))[Man(α1→6)Man(α1→3(6))]Man(β1→4)GlcNAc(β1→4) [Fuc(α1→6)]GlcNAc 1-,

### HexNAc(β1→4) [Fuc(α1→3(6))]GlcNAc(β1→2)Man(α1→6(3))[HexNAc(β1→4) [Fuc(α1→3(6))]GlcNAc(β1→2)Man(α1→3(6))]Man(β1→4)GlcNAc(β1→4) [Fuc(α1→6)]GlcNAcβ1-,

### HexNAc(β1→4) [Fuc(α1→3(6))]GlcNAc(β1→2)Man(α1→6(3))[Man(α1→6)Man(α1→6)Man(α1→3(6))]Man(β1→4) GcNAc(β1→4)[Fuc(α1→6)]GlcNAcβ1-,

### HexNAc(β1→4) [Fuc(α1→3(6))]GlcNAc(β1→2)Man(α1→6(3))[Man(α1→6(3))Man(α1→3(6))]Man(β1→6)GlcNAc(β1→4) [Fuc(α1→6)]GlcNAcβ1-+HexNAc,

### HexNAc(114) [Fuc(α1→3(6))]GlcNAc(β1→2)Man(α1→6(3))[NeuAc(α2→6(3))Gal(β1→4)GlcNAc(β1→2)Man(α1→3(6))]Man(β1→4)GlcNAc(β1→4) [Fuc(α1→6)]GlcNAcβ1-,

### Gal(β1→4) [Fuc(α1→3(6))]GlcNAc(β1→2)Man(α1→6) [Gal(α1→4)[Fuc(α1→3(6))]GlcNAc(α1→2)Man(α1→3)]Man(α1→4)GlcNAc(α1→4)[Fuc(α1→6)]GlcNAcβ1-,

### Gal(β1→4) [Fuc(α1→3(6))]GlcNAc(β1→2)Man(α1→6(3)) [Fuc(α1→3(6))GlcNAc(β1→2)Man(α1→3(6))]Man(β1→4)GlcNAc(β1→4)[Fuc(α1→6)]GlcNAcβ1-,

### HexNAc(β1→4) GcNAc(β1→2) Man (α1→6)[HexNAc(β1→4)GlcNAc(β1→2)Man(α1→3)]Man(β1→4)GlcNAc(β1→4)[Fuc(α1→6)]GlcNAcβ1-.

Still a further aspect of the current invention is a method for the determination of the binding of an immunoglobulin to a composition according to the invention obtained from HEK 293 cells, comprising the following steps:

i) providing an immunoglobulin to be analyzed,

ii) providing a composition of recombinant human Fc gamma receptor obtained from HEK 293 cells according to the invention,

iii) contacting the immunoglobulin with said composition of recombinant human Fc gamma receptor, and

iv) determining the binding of the immunoglobulin to said composition of recombinant human Fc gamma receptor.

Another aspect of the current invention is a method for the recombinant production of human Fc gamma receptor IIIa comprising the following steps:

providing a eukaryotic cell,

transfecting said provided eukaryotic cell with a heterologous nucleic acid encoding human Fc gamma receptor IIIa,

cultivating said transfected eukaryotic cell under conditions suitable for the expression of said human Fc gamma receptor IIIa,

recovering said recombinant human Fc gamma receptor IIIa from the eukaryotic cell or the cultivation medium,

whereby said recombinant human Fc gamma receptor IIIa is obtained as a composition comprising a mixture of at least two recombinant human Fc gamma receptor IIIa of SEQ ID NO: 1 differing in the N-linked oligosaccharide at amino acid position 163 of SEQ ID NO: 1.

Another aspect of the current invention is a composition comprising recombinant human Fc gamma receptor IIIa, whereby

a) the recombinant human Fc gamma receptor IIIa has the amino acid sequence of SEQ ID NO: 1 and has been expressed in CHO cells,
b) the composition comprises a mixture of at least two recombinant human Fc gamma receptor IIIa of SEQ ID NO: 1 differing in the N-linked oligosaccharide at amino acid position 163 of SEQ ID NO: 1,
whereby the at least two different oligosaccharides are selected from:
none,

### NeuAc(α2→6(3)Gal(β1→4)GlcNAc(β1→6)Man(α1→6)[Gal(β1→4)GlcNAc(β1→4)Man(α1→3)]Man(β1→4)GlcNAc(β1→4)[Fuc(α1→6)]GlcNAcβ1-,

### NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4)[Fuc(α1→6)]GlcNAcβ1-.

Another aspect of the invention is a method for the determination of the binding of an immunoglobulin to a composition according to the invention obtained from CHO cells, comprising the following steps

i) providing an immunoglobulin to be analyzed,

ii) providing a composition of recombinant human Fc gamma receptor obtained from CHO cells according to the invention,

iii) contacting the immunoglobulin with said composition of recombinant human Fc gamma receptor, and

iv) determining the binding of the immunoglobulin to said composition of recombinant human Fc gamma receptor.

### DESCRIPTION OF THE FIGURES

FIG. 1 Main N-linked oligosaccharides at position 163 of SEQ ID NO: 1.

FIG. 2 Plasmid map of pCLF60.

FIG. 3 BIAcore assay chromatogram of Fc gamma RIIIa expressed in HEK cells

FIG. 4 BIAcore assay chromatogram of Fc gamma RIIIa expressed in CHO cells

FIG. 5 Temporal representation of the scan event cycle.

FIG. 6 Total ion chromatogram for the RP-seporation of an Endoproteinase Glu-c/Chymotrypsin diges; A: total ion chromatogram; B: SID-Scan, Selected Ion Chromatogram for glycospecific Fragment Ions.

### DETAILED DESCRIPTION OF THE INVENTION

### A. Introduction and Embodiments

The current invention comprises a recombinant human Fc gamma receptor IIIa, with the amino acid sequence of SEQ ID NO: 1, and preferably further comprising at amino acid position 163 of SEQ ID NO: 1 one N-linked oligosaccharide.

In one embodiment the recombinant human Fc gamma receptor IIIa is expressed in HEK cells, preferably in HEK 293 cells. In another embodiment is the recombinant human Fc gamma receptor IIIa expressed in CHO cells.

In another embodiment the receptor, at amino acid position 75 of SEQ ID NO: 1 comprises one of the following N-linked oligosaccharides: NeuAc(α2→6(3)Gal(β1→4)GlcNAc(β1→6) [NeuAc(α2→6(3)Gal(β1→4)GlcNAc(β1→2)]Man(α1→6) [NeuAc(α2→6(3)Gal(β1→4)GlcNAc(β1→2) [NeuAc(α2→6(3)Gal(β1→4)GlcNAc(β1→4)]Man(α1→3)]Man(β1→4)GlcNAc(β1→4)[Fuc(α1→6)]GlcNAcβ1-, or

### NeuAc(α2→6(3)Gal(β1→4)GlcNAc(β1→6) [NeuAc(α2→6(3)Gal(β1→4)GlcNAc(β1→2)]Man(α1→6)[Gal(β1→4)GlcNAc(β1→2) [Gal(β1→4)GlcNAc(β1→4)]Man(α1→3)]Man(β1→4)GlcNAc(β1→4)[Fuc(α1→6)]GlcNAcβ1-, or

### NeuAc(α2→6(3)Gal(β1→4)GlcNAc(β1→6) [NeuAc(α2→6(3)Gal(β1→4)GlcNAc(β1→2)]Man(α1→6) [NeuAc(α2→6(3)Gal(β1→4)GlcNAc(β1→2) [Gal(β1→4)GlcNAc(β1→4)]Man(α1→3)]Man(β1→4)GlcNAc(β1→4) [Fuc(α1→6)]GlcNAcβ1-.

In another embodiment the receptor at amino acid position 46 of SEQ ID NO: 1 comprises one of the following N-linked oligosaccharides:

### Gal(β1→4)GlcNAc(β1→2)Man(α1→6(3))[GlcNAc(β1→2)Man(α1→3(6))]Man(β1→4)GlcNAc(β1→4) [Fuc(α1→6)]GlcNAcβ1-, or

### Man(α1→3(6))Man(α1→3(6))[Man(α1→3(6))]Man(α1→4)GcNAc(β1→4) [Fuc(α1→6)]GlcNAcβ1−, or

### Gal(β1→4)GlcNAc(β1→2)Man(α1→6)[Gal(β1→4)GlcNAc(β1→2)Man(α1→3)]Man(β1→4)GlcNAc(β1→4)[Fuc(α1→6)]GlcNAcβ1-, or

### GlcNAc(β1→2)Man(α1→6(3)) [Man (α1→3(6))]Man(α1→4)GcNAc(β1→4)[Fuc(α1→6)]GlcNAcβ1−+Hex, or

### Man(α1→3(6))Man(α1→3(6))[Man(α1→3(6))]Man(β1→4)GlcNAc(β1→4)GlcNAcβ1-.

In another embodiment the receptor at amino acid position 170 of SEQ ID NO: 1 comprises the following N-linked oligosaccharide:

### HexNAc(β1→4) [Fuc(α1→3(6))]GlcNAc(β1→2)Man(α1→6(3))[Gal(β1→4)GlcNAc(β1→2)Man(α1→3(6))]Man(β1→4)GlcNAc(β1→4) [Fuc(α1→6)]GlcNAcβ1-.

In another embodiment the receptor at one of the amino acid position 180 or 181 of SEQ ID NO: 1 comprises the following O-linked oligosaccharide:

### NeuAc(α2→6(3)Gal(β1→4) [NeuAc(α2→4(3)]HexNAc(β1→6)-.

In one embodiment the receptor has a phenylalanine at amino acid position 159.

The current invention also provides a method for determining the binding of an immunoglobulin to a recombinant Fc gamma receptor.

In one embodiment the Fc gamma receptor is conjugated to a solid phase. In another embodiment is the immunoglobulin conjugated to a solid phase.

In a further embodiment the conjugation of the Fc gamma receptor or the immunoglobulin is performed by chemically binding via N-terminal and/or ε-amino groups (lysine), ε-amino groups of different lysines, carboxy-, sulfhydryl-, hydroxyl- and/or phenolic functional groups of the amino acid backbone and/or sugar alcohol groups of the carbohydrate structure. In still another embodiment is the conjugation of the Fc gamma receptor or the immunoglobulin performed via a specific binding pair selected from the specific binding pairs (first component/second component):
Streptavidin or Avidin/biotin,
antibody/antigen,
lectin/polysaccharide,
steroid/steroid binding protein,
hormone/hormone receptor,
enzyme/substrate, or
immunoglobulin G/Protein A and/or G and/or L.

In a further embodiment of the method according to the invention the determining of the binding of the immunoglobulin to the Fc gamma receptor is achieved by surface plasmon resonance, acoustic resonance, fluorescence resonance energy transfer, immunoassays, total internal reflection, fiber optics, surface plasmon resonance enhanced fluorescence, or fluorescence activated cell sorting.

The invention also provides a method for determining the binding of an immunoglobulin to a composition of recombinant Fc gamma receptor obtained from HEK 293 cells.

In one embodiment of this method the recombinant human Fc gamma receptor is conjugated to a solid phase. In another embodiment the immunoglobulin is conjugated to a solid phase. In a further embodiment is the conjugation performed by chemically binding via N-terminal and/or ε-amino groups (lysine), ε-amino groups of different lysines, carboxy-, sulfhydryl-, hydroxyl- and/or phenolic functional groups of the amino acid backbone or sugar alcohol groups of the carbohydrate structure. In a still a further embodiment is that the conjugation to the solid phase is performed via a specific binding pair selected from the specific binding pairs (first component/second component):
Streptavidin or Avidin/biotin,
antibody/antigen,
lectin/polysaccharide,
steroid/steroid binding protein,
hormone/hormone receptor,
enzyme/substrate,
immunoglobulin G/Protein A and/or G and/or L.

In another embodiment the determination is selected from surface plasmon resonance, acoustic resonance, fluorescence resonance energy transfer, immunoassays, total internal reflection, fiber optics, surface plasmon resonance enhanced fluorescence, and fluorescence activated cell sorting, preferably by surface plasmon resonance. Another embodiment of the method is that the determination is by an immunoassay, either by a heterogeneous immunoassay or a sandwich immunoassay. In a further embodiment comprises the sandwich immunoassay a capture antibody immobilized to a solid phase and a detection antibody suited for direct or indirect detection. In one embodiment comprises the detection antibody a detectable label selected from chemoluminescent groups, fluorescent groups, luminescent metal complexes, enzymes, and radioisotopes.

In another embodiment comprises the detection antibody a first partner of a bioaffine binding pair selected from:

hapten or antigen/antibody,

biotin or biotin analogues such as aminobiotin, iminobiotin or desthiobiotin/avidin or Streptavidin,

sugar/lectin,

nucleic acid or nucleic acid analogue/complementary nucleic acid, and

receptor/ligand.

The invention also provides a method for the recombinant production of Fc gamma receptor IIIc via transfection of an eukaryotic cell with a heterologous nucleic acid encoding Fc gamma receptor IIIa, wherein the recombinant Fc gamma receptor composition obtained comprises a mixture of at least two recombinant Fc gamma receptor IIIa of SEQ ID No: 1 differing in N-linked oligosaccharide at amino acid position 163 of SEQ ID NO: 1.

In one embodiment the eukaryotic cell, is a HEK 293 cell, and said at least two different oligosaccharides are selected from:

none,

### HexNAc(β1→4) [Fuc(α1→3(6))]GlcNAc(β1→2)Man(α1→6(3))[HexNAc(β1→4)GlcNAc(β1→2)Man(α1→3(6))]Man(β1→4)GlcNAc(β1→4) [Fuc(α1→6)]GlcNAcβ1-,

### HexNAc(β1→4) [Fuc(α1→3(6))]GlcNAc(β1→2)Man(α1→6(3))[Man(α1→6)Man(α1→3(6))]Man(β1→4)GlcNAc(β1→4) [Fuc(α1→6)]GlcNAc 1-,

### HexNAc(β1→4) [Fuc(α1→3(6))]GlcNAc(β1→2)Man(α1→6(3))[HexNAc(β1→4) [Fuc(α1→3(6))]GlcNAc(β1→2)Man(α1→3(6))]Man(β1→4)GlcNAc(β1→4) [Fuc(α1→6)]GlcNAcβ1-,

### HexNAc(β1→4) [Fuc(α1→3(6))]GlcNAc(β1→2)Man(α1→6(3))[Man(α1→6)Man(α1→6)Man(α1→3 (6))]Man(β1→4) GlcNAc(β1→14)[Fuc(α1→6)]GlcNAcβ1-,

### HexNAc(β1→4) [Fuc(α1→3(6))]GlcNAc(β1→2)Man(α1→6(3))[Man(α1→6(3))Man(α1→3(6))]Man(β1→6)GlcNAc(β1→4) [Fuc(α1→6)]GlcNAcβ1-+HexNAc,

### HexNAc(β1→4) [Fuc(α1→3(6))]GlcNAc(β1→2)Man(α1→6(3))[NeuAc(α2→6(3))Gal(β1→4)GlcNAc(β1→2)Man(α1→3(6))]Man(1-4)GlcNAc(β1→4)[Fuc(α1→6)]GlcNAcβ1-,

### Gal(β1→4) [Fuc(α1→3(6))]GlcNAc(β1→2)Man(α1→6) [Gal(α1→4)[Fuc(α1→3(6))]GlcNAc(α1→2)Man(α1→3)]Man(α1→4)GlcNAc(α1→4)[Fuc(α1→6)]GlcNAcβ1-,

### Gal(β1→4) [Fuc(α1→3(6))]GlcNAc(β1→2)Man(α1→6(3)) [Fuc(α1→3(6))GlcNAc(β1→2)Man(α1→3(6))]Man(β1→4)GlcNAc(β1→4) [Fuc(α1→6)]GlcNAcβ1−,

### HexNAc(β1→4) GcNAc(β1→2)Man (α1→6)[HexNAc(β1→4)GlcNAc(β1→2)Man(α1→3)]Man(β1→4)GlcNAc(β1→4)[Fuc(α1→6)]GlcNAcβ1-.

In a different embodiment is the eukaryotic cell a CHO cell and said at least two different oligosaccharides are selected from:

none,

### NeuAc(α2→6(3)Gal(β1→4)GlcNAc(β1→6)Man(α1→6) [Gal(β1→4)GlcNAc(β1→4)Man(α1→3)]Man(β1→4)GlcNAc(β1→4) [Fuc(α1→6)]GlcNAcβ1-,

### NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4)[Fuc(α1→6)]GlcNAcβ1-.

The invention also provides a method for determining the binding of an immunoglobulin to a composition of recombinant human Fc gamma receptor obtained from CHO cells.

In one embodiment the recombinant human Fc gamma receptor is conjugated to a solid phase.

In a further embodiment the immunoglobulin is conjugated to a solid phase. In another embodiment the conjugation is performed by chemically binding via N-terminal and/or ε-amino groups (lysine), ε-amino groups of different lysines, carboxy-, sulfhydryl-, hydroxyl- and/or phenolic functional groups of the amino acid backbone or sugar alcohol groups of the carbohydrate structure. In still a further embodiment is the conjugation to the solid phase performed via a specific binding pair selected from the group of specific binding pairs comprising (first component/second component):
Streptavidin or Avidin/biotin,
antibody/antigen,
lectin/polysaccharide,
steroid/steroid binding protein,
hormone/hormone receptor,
enzyme/substrate,
immunoglobulin G/Protein A and/or G and/or L.

In another embodiment of this aspect of the invention the determination is by a method selected from surface plasmon resonance, acoustic resonance, fluorescence resonance energy transfer, immunoassays, total internal reflection, fiber optics, surface plasmon resonance enhanced fluorescence, and fluorescence activated cell sorting, preferably by surface plasmon resonance. In another embodiment is the determination by an immunoassay. In still a further embodiment is the immunoassay a heterogeneous immunoassay. In a further embodiment is the immunoassay a sandwich immunoassay. Another embodiment is that said sandwich immunoassay comprises a capture antibody immobilized to a solid phase and a detection antibody suited for direct or indirect detection. In a further embodiment comprises the detection antibody a detectable label selected from chemoluminescent groups, fluorescent groups, luminescent metal complexes, enzymes, and radioisotopes. In still another embodiment comprises the detection antibody a first partner of a bioaffine binding pair selected from:

hapten or antigen/antibody,

biotin or biotin analogues such as aminobiotin, iminobiotin or desthiobiotin/avidin or Streptavidin,

sugar/lectin,

nucleic acid or nucleic acid analogue/complementary nucleic acid, and

receptor/ligand.

Methods and techniques known to a person skilled in the art, which are useful for carrying out the current invention, are described e.g. in Ausubel, F. M., ed., Current Protocols in Molecular Biology, Volumes I to III (1997), Wiley and Sons; Sambrook et al., Molecular Cloning: A Laboratory Manual, Second Edition, Cold Spring Harbor Laboratory Press, Cold Spring Harbor, N.Y. (1989), hereby incorporated by reference in its entirety.

### B. Definitions

A “nucleic acid” as used herein, refers to a naturally occurring or partially or fully non-naturally occurring nucleic acid encoding a polypeptide which can be produced recombinantly. A nucleic acid is a polymeric molecule comprising as monomers nucleotides. The nucleic acid can be build up of DNA-fragments which are either isolated or synthesized by chemical means. The nucleic acid can be integrated into another nucleic acid, e.g. in an expression plasmid or the genome/chromosome of a eukaryotic host cell. The term “plasmid” includes shuttle and expression vectors. Typically, a plasmid will also comprise a prokaryotic propagation unit comprising an origin of replication (e.g. the ColE1 origin of replication) and a selectable marker (e.g. ampicillin or tetracycline resistance gene), for replication and selection, respectively, of the plasmid in bacteria/prokaryotes.

An “expression cassette” refers to a nucleic acid that contains the elements necessary for expression and secretion of at least the contained structural gene in a cell.

A nucleic acid is likewise characterized by its nucleic acid sequence consisting of individual nucleotides or/and by an amino acid sequence encoded by the nucleic acid.

A “gene” denotes a segment e.g. on a chromosome or on a plasmid which is necessary for the expression of a peptide, polypeptide, or protein. Beside the coding region the gene comprises other functional elements including a promoter, introns, and terminators.

A “structural gene” denotes the polypeptide encoding region of a gene without a signal sequence.

A “resistance gene” or a “selectable marker”, which is used interchangeably within this application, is a gene/nucleic acid that allows cells carrying it to be specifically selected for or against, in the presence of a corresponding selection agent. A useful positive resistance gene is an antibiotic resistance gene. This selectable marker allows the host cell transformed with the corresponding nucleic acid to be positively selected for in the presence of the corresponding antibiotic. A non-transformed host cell would not be capable to grow and/or survive in the presence of the corresponding selection agent, i.e. under selective culture conditions. Selectable markers can be positive, negative, or bifunctional. Positive selectable markers allow for selection of cells carrying the marker, whereas negative selectable markers allow cells carrying the marker to be selectively eliminated. Typically, a selectable marker will confer resistance to a drug or compensate for a metabolic or catabolic defect in the host cell. Selectable markers useful with eukaryotic cells include, e.g., the genes for aminoglycoside phosphotransferase (APH), such as the hygromycin phosphotransferase (hyg), neomycin and G418 APH, dihydrofolate reductase (DHFR), thymidine kinase (tk), glutamine synthetase (GS), asparagine synthetase, tryptophan synthetase (indole), histidinol dehydrogenase (histidinol D), and genes encoding resistance to puromycin, bleomycin, phleomycin, chloramphenicol, Zeocin, and mycophenolic acid. Further selectable marker genes are reported e.g. in WO 92/08796 and WO 94/28143.

To produce a secreted polypeptide, the structural gene of interest also comprises a DNA segment that encodes a signal sequence/leader peptide. The signal sequence directs the newly synthesized polypeptide to and through the membrane of the Endoplasmic Reticulum (ER) where the polypeptide can be routed for secretion. The signal sequence is cleaved off by a signal peptidases during the protein crosses the ER membrane. As for the function of the signal sequence the recognition by the host cell's secretion machinery is essential. Therefore the used signal sequence has to be recognized by the host cell's proteins and enzymes of the secretion machinery.

Translational regulatory elements include a translational initiation (AUG) and stop codon (TAA, TAG or TGA). An internal ribosome entry site (IRES) can be included in some constructs.

The term “expression” as used herein refers to transcription and/or translation of a nucleic acid occurring within a host cell. The level of transcription of a desired nucleic acid in a host cell can be determined on the basis of the amount of corresponding mRNA that is present in said cell. For example, mRNA transcribed from a selected nucleic acid can be quantitated by PCR or by Northern hybridization (see e.g. Sambrook et al., Molecular Cloning: A Laboratory Manual, Cold Spring Harbor Laboratory Press (1989)). Protein encoded by a selected nucleic acid can be quantitated by various methods, e.g. by ELISA, by assaying for the biological activity of the protein, or by employing assays that are independent of such activity, such as Western blotting or radioimmunoassay, using antibodies that recognize and bind to the protein (see e.g. Sambrook et al., 1989, supra).

A “host cell” refers to a cell into which the gene/nucleic acid encoding a polypeptide of the invention is introduced. Host cell includes both prokaryotic cells used for propagation of the plasmids/vectors, and eukaryotic cells for expression of the structural gene. Typically, the eukaryotic cells are mammalian cells.

A “polypeptide” is a polymer of amino acid residues joined by peptide bonds, whether produced naturally or synthetically. Polypeptides of less than about 20 amino acid residues may be referred to as “peptides.” Polypeptides comprising one or more polypeptide chains or comprising a single amino acid chain of a length of 100 amino acids or more may be referred to as “proteins”.

A “protein” is a macromolecule comprising either a single polypeptide chain of a length of 100 amino acids or more or two or more polypeptides. A protein may also comprise non-peptidic components, such as carbohydrate groups. Carbohydrate groups and other non-peptidic components may be added to a protein by the cell in which the protein is produced, and may vary with the type of cell. Proteins are defined herein in terms of their amino acid backbone structures/amino acid seqeuences; additions such as carbohydrate groups are generally not specified, but may be present nonetheless.

“Heterologous DNA” or “heterologous polypeptide” refers to a DNA molecule or a polypeptide, or a population of DNA molecules or a population of polypeptides, that do not exist naturally within a given host cell. DNA molecules heterologous to a particular host cell may contain DNA derived from the host cell species (i.e. endogenous DNA) so long as that host DNA is combined with non-host DNA (i.e. exogenous DNA). For example, a DNA molecule containing non-host DNA encoding a polypeptide operably linked to host DNA comprising a promoter is considered to be a heterologous DNA molecule. Conversely, a heterologous DNA molecule can comprise an endogenous structural gene operably linked with an exogenous promoter.

A peptide or polypeptide encoded by a non-host DNA molecule is a “heterologous” peptide or polypeptide.

A “cloning vector” is a nucleic acid molecule, such as a plasmid, cosmid, phagemid, or bacterial artificial chromosome (BAC), which has the capability of replicating autonomously in a host cell. Cloning vectors typically contain one or a small number of restriction endonuclease recognition sites that allow insertion of a nucleic acid in a determinable fashion without loss of an essential biological function of the vector, as well as nucleotide sequences encoding a selectable marker that is suitable for use in the identification and selection of cells transformed with the cloning vector. Resistance genes typically include genes that provide tetracycline resistance or ampicillin resistance.

An “expression plasmid” is a nucleic acid encoding a polypeptiden to be expressed in a host cell. Typically, an expression plasmid comprises a prokaryotic plasmid propagation unit, e.g. for E. coli, comprising an origin of replication, and a nucleic acid encoding a selectable marker, an eukaryotic selectable marker, and one or more expression cassettes for the expression of the structural gene(s) of interest each comprising a promoter, a structural gene, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

An “isolated polypeptide” is a polypeptide that is essentially free from contaminating cellular components, such as carbohydrate, lipid, or other proteinaceous impurities associated with, i.e. not covalently bound to, the polypeptide in nature. Typically, a preparation of isolated polypeptide contains the polypeptide in a highly purified form, i.e. at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

The term “immunoglobulin” refers to a protein consisting of one or more polypeptides substantially encoded by immunoglobulin genes. The recognized immunoglobulin genes include the different constant region genes as well as the myriad immunoglobulin variable region genes. Immunoglobulins may exist in a variety of formats, including, for example, Fv, Fab, and F(ab)₂ as well as single chain (scFv) (e.g. Huston, J. S., et al., Proc. Natl. Acad. Sci. USA 85 (1988) 5879-5883; Bird, R. E., et al., Science 242 (1988) 423-426; in general, Hood et al., Immunology, Benjamin N.Y., 2nd edition (1984); and Hunkapiller, T. and Hood, L., Nature 323 (1986) 15-16).

An “immunoglobulin fragment” denotes a polypeptide comprising at least the constant domains of a chain of an immunoglobulin, i.e. the C_{H}1 domain, the hinge-region, the C_{H}2 domain, the C_{H}3 domain, and optionally the C_{H}4 domain of a heavy chain of an immunoglobulin or the C_{L} domain of a light chain of an immunoglobulin. Also comprised are derivatives and variants thereof. Additionally a variable domain, in which one or more amino acids or amino acid regions are deleted, may be present.

The term “amino acid” as used within this application comprises alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

The terms “glycostructure”, “glycosylation” and “glycosylation pattern” which are used interchangeably within this application comprises all the oligosaccharides which are attached to a specified amino acid residue in a recombinantly produced polypeptide. Due to the glycosylation heterogeneity of a cell, a recombinantly produced polypeptide comprises not only a single, defined N- or O-linked oligosaccharide at a specified amino acid residue, but is a mixture of polypeptides each having the same amino acid sequence but comprising different oligosaccharides at said specified amino acid position. Thus, the above terms denote a group of oligosaccharides that are attached to a specified amino acid position of a recombinantly produced polypeptide, i.e. the heterogeneity of the attached oligosaccharide. The term “oligosaccharide” as used within this application denotes a polymeric saccharide comprising two or more covalently linked monosaccharide units.

### C. Detailed Description

The first aspect of the current invention is recombinant human Fc gamma receptor IIIa, that has the amino acid sequence of SEQ ID NO: 1, and that is expressed and isolated from HEK 293 cells, and that comprises at amino acid position 163 of SEQ ID NO: 1 one of the following N-linked oligosaccharides:

none, or

### HexNAc(β1→4) [Fuc(α1→3(6))]GlcNAc(β1→2)Man(α1→6(3)) [HexNAc(β1→4)GlcNAc(β1→2)Man(α1→3(6))]Man(β1→4)GlcNAc(β1→4) [Fuc(α1→6)]GlcNAcβ1-, or

### HexNAc(β1→4) [Fuc(α1→3(6))]GlcNAc(β1→2)Man(α1→6(3)) [Man(α1→6)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3)) [HexNAc(β1→4)[Fuc(α1→3(6))]GlcNAc(β1→2)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3)) [Man(α1→6)Man(α1→6)Man(α1→3(6))]Man(β1→4)GlcNAc(β1→4) [Fuc(α1→6)]GlcNAcβ1-, or

### HexNAc(β1→4) [Fuc(α1→3(6))]GlcNAc(β1→2)Man(α1→6(3))[Man(α1→6(3))Man(α1→3(6))]Man(α1→6)GlcNAc(β1→4) [Fuc(α1→6)]GlcNAcβ1-+HexNAc, or

### HexNAc(β1→4) [Fuc(13 (6))]GlcNAc(β1→2) Man(16 (3))[NeuAc(α2→6(3))Gal(α1→4)GlcNAc(12)Man(α1→3(6))]Man(β1→4)GlcNAc(α1→4) [Fuc(α1→6)]GlcNAcβ1-, or

### Gal(β1→4) [Fuc(α1→3(6))]GlcNAc(β1→2)Man(α1→6)[Gal(α1→4)[Fuc(α1→3(6))]GlcNAc(α1→2)Man(α1→3)]Man(β1→4)GlcNAc(β1→4)[Fuc(α1→6)]GlcNAcβ1-, or

### Gal(β1→4) [Fuc(α1→3(6))]GlcNAc(β1→2)Man(α1→6(3))[Fuc(α1→3(6))GlcNAc(β1→2)Man(α1→3(6))]Man(β1→4)GlcNAc(β1→4)[Fuc(α1→6)]GlcNAcβ1-, or

### HexNAc(β1→4)GlcNAc(β1→2)Man(α1→6)[HexNAc(β1→4)GlcNAc(β1→2)Man(α1→3)]Man(β1→4)GlcNAc(β1→4)[Fuc(α1→6)]GlcNAcβ1-.

SEQ ID NO: 1 comprises the following amino acid sequence given in one-letter code: N-terminus-

(SEQ ID NO:1)

MRTEDLPKAVVFLEPQWYRVLEKDSVTLKCQGAYSPEDNSTQWFHNESLI
SSQASSYFIDAATVDDSGEYRCQTNLSTLSDPVQLEVHIGWLLLQAPRWV
FKEEDPIHLRCHSWKNTALHKVTYLQNGKGRKYFHHNSDFYIPKATLKDS
GSYFCRGLVGSKNVSSETVNITITQGLAVSTISSFFPPGYQ-C-
terminus

SEQ ID NO: 1 denotes the extracellular domain of the human Fc gamma Receptor Type III, whose complete amino acid sequence including the signal peptide is given in SEQ ID NO: 2 (see also e.g. Swiss-Prot entry P08637).

For the notation of the different N- or O-linked oligosaccharides in the current invention the individual sugar residues are listed from the non-reducing end to the reducing end of the oligosaccharide molecule. The longest sugar chain was chosen as basic chain for the notation. The reducing end of an N- or O-linked oligosaccharide is the sugar residue, which is directly bound to the amino acid of the amino acid backbone of the receptor, whereas the end of an N- or O-linked oligosaccharide, which is located at the opposite terminus as the reducing end of the basic chain, is termed non-reducing end.

The second aspect of the current invention is recombinant human Fc gamma receptor IIIa, that has the amino acid sequence of SEQ ID NO: 1, that is expressed and isolated from CHO cells, and that comprises at amino acid position 163 of SEQ ID NO: 1 one of the following N-linked oligosaccharides:

none, or

### NeuAc(α2→6(3)Gal(β1→4)GlcNAc(β1→6)Man(α1→6) [Gal(β1→4)GlcNAc(β1→4)Man(α1→3)]Man(β1→4)GlcNAc(β1→4) [Fuc(α1→6)]GlcNAcβ1-, or

### NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4) [Fuc(α1→6)]GlcNAcβ1-.

Due to the fact that in SEQ ID NO: 1 five N-glycosylation sites can be found, the glycosylation pattern can neither be studied by whole protein mass analysis nor by analyzing the glycans only. For example the glycosylation sites at position 39 and position 46 of SEQ ID NO:1, respectively, likewise the glycosylation sites at position 163 and position 170 of SEQ ID NO: 1, respectively, are very close together. For the digestion to separate these pairs of glycosylation sites special enzymes are required.

For the digestion, i.e. enzymatic cleavage, of polypeptides different endoproteases can be employed, such as e.g. Glu-C (an endoprotease from Staphylococcus aureus (Protease V8), specifity for a C-terminal glutamic acid residue), Sialidase (a neuramidase catalyzing the hydrolysis of terminal acetyl neuraminic acid residues), and Chymotrypsin (an endopeptidase cleaving peptide bonds N-terminal to tyrosine, tryptophan, and phenylalanine).

It has been surprisingly found in the current invention that a combined digestion with Glu-C and Chymotrypsin, optionally with additional Sialidase, gives best results for the analysis of the glycosylation, i.e. the glycostructure, at position 163 of SEQ ID NO: 1. It has been found that the analysis of the glycosylation can be performed by reduction, alkylation, and enzymatic cleavage of the polypeptide, followed by reverse phase (RP)HPL-chromatography with MS detection (SID scan and MS-MS coupling) using electrospray ionization and a LTQ FT mass spectrometer (linear trap mass spectrometer with five MS/MS scans following each full MS scan). The glycosylation pattern is very complex and it was surprisingly found that the structure elucidation can be done by MS/MS analysis.

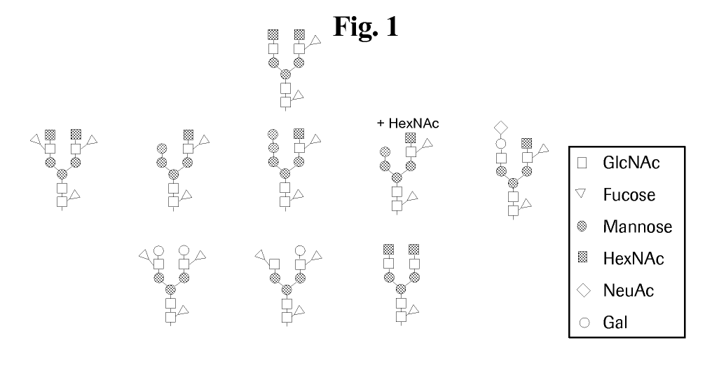

With the above reported approach the following main N-linked oligosaccharides at position 163 of SEQ ID NO: 1 have been identified (see also FIG. 1) for the first aspect of the current invention (□=N-acetylglucosamine [GlcNAc], [symbol] =fucose [Fuc], [symbol] =mannose [Man], [symbol] =N-acetylhexose [HexNAc], [symbol] =N-acetylneuraminic acid [NeuAc], ◯=galactose [Gal]):

| | | |
|---|---|---|
| 1 | 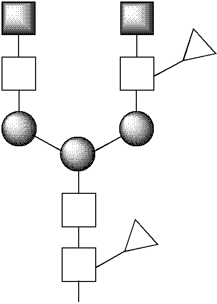 | HexNAc(β1→4)[Fuc(α1→3(6))]GlcNAc(β1→2)Man(α1→6(3))[HexNAc(β1→4)GlcNAc(β1→2)Man(α1→3(6))]Man(β1→4)GlcNAc(β1→4)[Fuc(α1→6)]GlcNAcβ1-, |
| 2 | 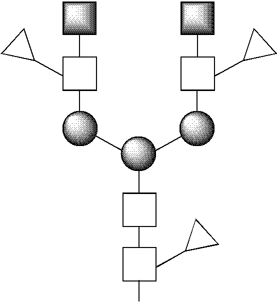 | HexNAc(β1→4)[Fuc(α1→3(6))]GlcNAc(β1→2)Man(α1→6(3))[HexNAc(β1→4)[Fuc(α1→3(6))]GlcNAc(β1→2)Man(α1→3(6))]Man(β1→4)GlcNAc(β1→4)[Fuc(α1→6)]GlcNAcβ1-, |
| 3 | 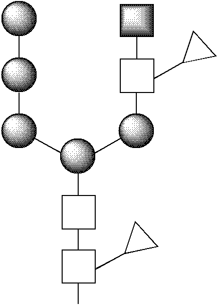 | HexNAc(β1→4)[Fuc(α1→3(6))]GlcNAc(β1→2)Man(α1→6(3))[Man(α1→6)Man(α1→6)Man(α1→3(6))]Man(β1→4)GlcNAc(β1→4)[Fuc(α1→6)]GlcNAcβ1-, |
| 4 | 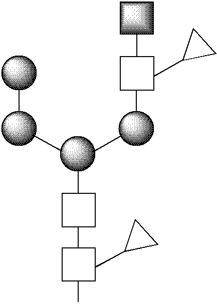 | HexNAc(β1→4)[Fuc(α1→3(6))]GlcNAc(β1→2)Man(α1→6(3))[Man(α1→6)Man(α1→3(6))]Man(β1→4)GlcNAc(β1→4)[Fuc(α1→6)]GlcNAcβ1-, |
| 5 | 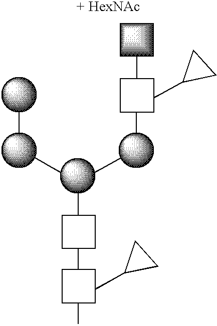 | HexNAc(β1→4)[Fuc(α1→3(6))]GlcNAc(β1→2)Man(α1→6(3))[Man(α1→6(3))Man(α1→3(6))]Man(β1→6)GlcNAc(β1→4)[Fuc(α1→6)]GlcNAcβ1-, +HexNAc |
| 6 | 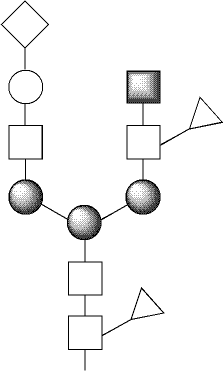 | HexNAc(β1→4)[Fuc(α1→3(6))]GlcNAc(β1→2)Man(α1→6(3))[NeuAc(α2→6(3))Gal(β1→4)GlcNAc(β1→2)Man(α1→3(6))]Man(β1→4)GlcNAc(β1→4)[Fuc(α1→6)]GlcNAcβ1-, |
| 7 | 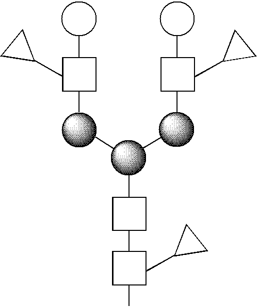 | Gal(β1→4)[Fuc(α1→3(6))]GlcNAc(β1→2)Man(α1→6)[Gal(α1→4)[Fuc(α1→3(6))]GlcNAc(β1→2)Man(α1→3)]Man(β1→4)GlcNAc(β1→4)[Fuc(α1→6)]GlcNAcβ1-, |
| 8 | 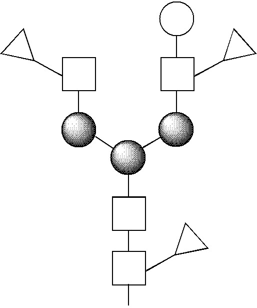 | Gal(β1→4)[Fuc(α1→3(6))]GlcNAc(β1→2)Man(α1→6(3))[Fuc(α1→3(6))GlcNAc(β1→2)Man(α1→3(6))]Man(β1→4)GlcNAc(β1→4)[Fuc(α1→6)]GlcNAcβ1-, |
| 9 | 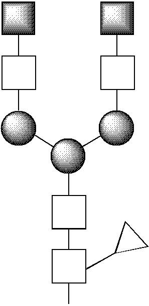 | HexNAc(β1→4)GlcNAc(β1→2)Man(α1→6)[HexNAc(β1→4)GlcNAc(β1→2)Man(α1→3)]Man(β1→4)GlcNAc(β1→4)[Fuc(α1→6)]GlcNAcβ1-. |

The recombinant human Fc gamma Receptor IIIa of the current invention is a mixture of differently glycosylated molecules comprising the above identified N-linked oligosaccharides with different relative frequency. The recombinant receptor will also to a certain extent be not glycosylated at one or more glycosylation sites referred to in this application. Thus, the glycosylation profile presented herein is an average glycosylation profile. And, therefore, one aspect of the current invention is a composition comprising recombinant human Fc gamma receptor IIIa, wherein the recombinant human Fc gamma receptor IIIa has the amino acid sequence of SEQ ID NO: 1 and has been expressed and/or has been isolated from HEK 293 cells, and wherein the composition comprises a mixture of at least two recombinant human Fc gamma receptor IIIa of SEQ ID NO: 1 differing in the N-linked oligosaccharide at amino acid position 163 of SEQ ID NO: 1, whereby said at least two different oligosaccharide is selected from:

none, or

### HexNAc(β1→4) [Fuc(α1→3(6))]GlcNAc(β1→2)Man(α1→6(3)) [HexNAc(β1→4)GlcNAc(β1→2)Man(α1→3(6))]Man(β1→4)GlcNAc(β1→4) [Fuc(α1→6)]GlcNAcβ1-, or

### HexNAc(β1→4) [Fuc(α1→3(6))]GlcNAc(β1→2)Man(α1→6(3)) [Man(α1→6)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3)) [HexNAc(β1→4)[Fuc(α1→3(6))]GlcNAc(β1→2)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3))[Man(α1→6)Man(α1→6)Man(α1→3(6))]Man(β1→4)GlcNAc(β1→4) [Fuc(α1→6)]GlcNAcβ1-, or

### HexNAc(β1→4) [Fuc(α1→3(6))]GlcNAc(β1→2)Man(α1→6(3)) [Man(α1→6(3))Man(α1→3(6))]Man(α1→6)GlcNAc(β1→4) [Fuc(α1→6)]GlcNAcβ1-+HexNAc, or

### HexNAc(β1→4)[Fuc(α1→3 (6))]GlcNAc(β1→2) Man(α1→6(3))[NeuAc(α2→6 (3))Gal(α1→4) GcNAc(β1→2)Man(α1→3(6))]Man(β1→4)GlcNAc(α1→4) [Fuc(α1→6)]GlcNAcβ1-, or

### Gal(β1→4)[Fuc(α1→3(6))]GlcNAc(β1→2)Man(α1→6)[Gal(α1→4)[Fuc(α1→3(6))]GlcNAc(β1→2)Man(α1→3)]Man(β1→4)GlcNAc(β1→4)[Fuc(α1→6)]GlcNAcβ1-, or

### Gal(β1→4) [Fuc(α1→3(6))]GlcNAc(β1→2)Man(α1→6(3)) [Fuc(α1→3(6))GlcNAc(β1→2)Man(α1→3(6))]Man(β1→4)GlcNAc(β1→4) [Fuc(α1→6)]GlcNAcβ1-, or

### HexNAc(β1→4) GcNAc(β1→2)Man(α1→6) [HexNAc(β1→4)GlcNAc(β1→2)Man(α1→3)]Man(β1→4)GlcNAc(β1→4)[Fuc(α1→6)]GlcNAcβ1-.

The main N-linked oligosaccharide at position 163 of SEQ ID NO: 1 of recombinant human Fc gamma Receptor IIIa expressed in HEK cells is oligosaccharide number 1, the secondary N-linked oligosaccharide is oligosaccharide number 4.

With the above identified approach also N- and O-linked oligosaccharides at other amino acid positions of SEQ ID NO: 1 have been identified.

In one embodiment of the invention comprises the receptor expressed in HEK 293 cells at amino acid position 75 of SEQ ID NO: 1 none or one of the following N-linked oligosaccharides:

| | | |
|---|---|---|
| 10 | 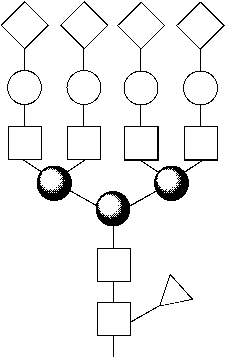 | NeuAc(α2→6(3)Gal(β1→4)GlcNAc(β1→6)[NeuAc(α2→6(3)Gal(β1→4)GlcNAc(β1→2)]Man(α1→6)[NeuAc(α2→6(3)Gal(β1→4)GlcNAc(β1→2)[NeuAc(α2→6(3)Gal(β1→4)GlcNAc(β1→4)]Man(α1→3)]Man(β1→4)GlcNAc(β1→4)[Fuc(α1→6)]GlcNAcβ1-, |
| 11 | 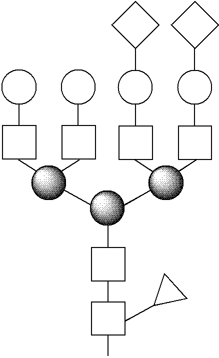 | NeuAc(α2→6(3)Gal(β1→4)GlcNAc(β1→6)[NeuAc(α2→6(3)Gal(β1→4)GlcNAc(β1→2)]Man(α1→6)[Gal(β1→4)GlcNAc(β1→2)[Gal(β1→4)GlcNAc(β1→4)]Man(α1→3)]Man(β1→4)GlcNAc(β1→4)[Fuc(α1→6)]GlcNAcβ1-, or- |
| 12 | 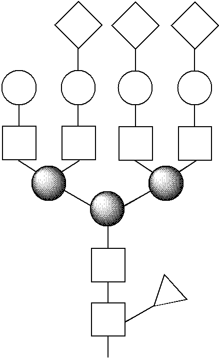 | NeuAc(α2→6(3)Gal(β1→4)GlcNAc(β1→6)[NeuAc(α2→6(3)Gal(β1→4)GlcNAc(β1→2)]Man(α1→6)[NeuAc(α2→6(3)Gal(β1→4)GlcNAc(β1→2)[Gal(β1→4)GlcNAc(β1→4)]Man(α1→3)]Man(β1→4)GlcNAc(β1→4)[Fuc(α1→6)]GlcNAcβ1-. |

The main N-linked oligosaccharide at position 75 of SEQ ID NO: 1 of recombinant human Fc gamma Receptor IIIa expressed in HEK 293 cells is oligosaccharide number 10. The secondary N-linked oligosaccharides at position 75 of SEQ ID NO: 1 is one of the oligosaccharides of number 11, or 12.

In another embodiment comprises the receptor expressed in HEK 293 cells at amino acid position 46 of SEQ ID NO: 1 none or one of the following N-linked oligosaccharides:

| | | |
|---|---|---|
| 13 | 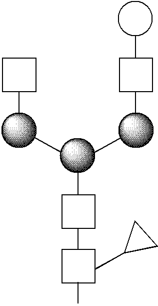 | Gal(β1→4)GlcNAc(β1→2)Man(α1→6(3))[GlcNAc(β1→2)Man(α1→3(6))]Man(β1→4)GlcNAc(β1→4)[Fuc(α1→6)]GlcNAcβ1-, |
| 14 | 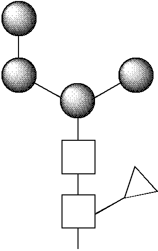 | Man(α1→3(6))Man(α1→3(6))[Man(α1→3(6))]Man(β1→4)GlcNAc(β1→4)[Fuc(α1→6)]GlcNAcβ1-, |
| 15 | 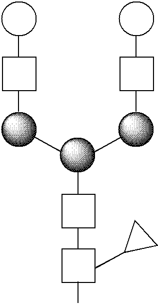 | Gal(β1→4)GlcNAc(β1→2)Man(α1→6)[Gal(β1→4)GlcNAc(β1→2)Man(α1→3)]Man(β1→4)GlcNAc(β1→4)[Fuc(α1→6)]GlcNAcβ1-, |
| 16 | 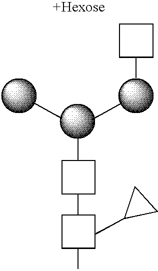 | GlcNAc(β1→2)Man(α1→6(3))[Man(α1→3(6))]Man(β1→4)GlcNAc(β1→4)[Fuc(α1→6)]GlcNAcβ1-, +Hex, or |
| 17 | 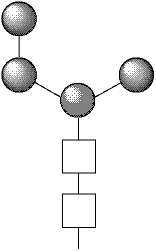 | Man(α1→3(6))Man(α1→3(6))[Man(α1→3(6))]Man(β1→4)GlcNAc(β1→4)GlcNAcβ1-. |

In another embodiment comprises the receptor expressed in HEK 293 cells at amino acid position 170 of SEQ ID NO: 1 none or the following N-linked oligosaccharide:

| | | |
|---|---|---|
| 18 | 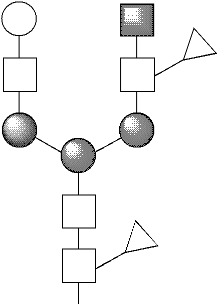 | HexNAc(β1→4)[Fuc(α1→3(6))]GlcNAc(β1→2)Man(α1→6(3))[Gal(β1→4)GlcNAc(β1→2)Man(α1→3(6))]Man(β1→4)GlcNAc(β1→4)[Fuc(α1→6)]GlcNAcβ1-. |

In another embodiment comprises the receptor expressed in HEK 293 cells at amino acid position 180 or 181 of SEQ ID NO: 1 none or the following O-linked oligosaccharide:

| | | |
|---|---|---|
| 19 | 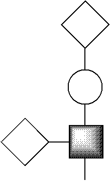 | NeuAc(α2→6(3)Gal(β1→4)[NeuAc(α2→4(3)]HexNAc(β1→6)-. |

In one embodiment has the receptor a phenylalanine at amino acid position 159 of SEQ ID NO: 1.

The term “HexNAc” as used within this application denotes an N-acetylated galactosamine or glucosamine sugar residue (GalNAc or GlcNAc).

A further aspect of the current invention is recombinant human Fc gamma receptor IIIa, that has the amino acid sequence of SEQ ID NO: 1, that is expressed and isolated from CHO cells, and that comprises at amino acid position 163 of SEQ ID NO: 1 none or one of the following N-linked oligosaccharides:

| | | |
|---|---|---|
| 20 | 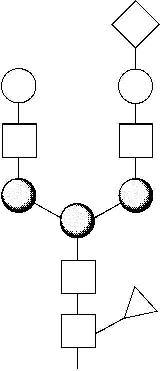 | NeuAc(α2→6(3)Gal(β1→4)GlcNAc(β1→6)Man(α1→6)[Gal(β1→4)GlcNAc(β1→4)Man(α1→3)]Man(β1→4)GlcNAc(β1→4)[Fuc(α1→6)]GlcNAcβ1-, or |
| 21 | 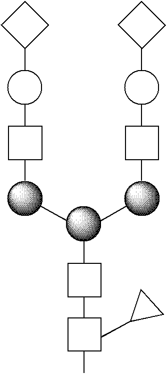 | NeuAc(α2→6(3)Gal(β1→4)GlcNAc(β1→6)[NeuAc(α2→6(3)Gal(β1→4)GlcNAc(β1→4)Man(α1→3)]Man(β1→4)GlcNAc(β1→4)[Fuc(α1→6)]GlcNAcβ1-. |

Another aspect of the current invention is a composition comprising recombinant human Fc gamma receptor IIIa, wherein the recombinant human Fc gamma receptor IIIa has the amino acid sequence of SEQ ID NO: 1 and has been expressed and/or isolated from CHO cells, and wherein the composition comprises a mixture of at least two recombinant human Fc gamma receptor IIIa of SEQ ID NO: 1 differing in the N-linked oligosaccharide at amino acid position 163 of SEQ ID NO: 1, whereby said at least two different oligosaccharides are selected from:

none, or

### NeuAc(α2→6(3)Gal(β1→4)GlcNAc(β1→6)Man(α1→6) [Gal(β1→4)GlcNAc(β1→4)Man(α1→3)]Man(β1→4)GlcNAc(β1→4) [Fuc(α1→6)]GlcNAcβ1-, or

### NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4) [Fuc(α1→6)]GlcNAcβ1-.

A further aspect of the invention is a method for the determination of the binding of an immunoglobulin to a recombinant Fc gamma receptor according to the invention, comprising the following steps

i) providing an immunoglobulin to be analyzed,
ii) providing a recombinant Fc gamma receptor according to the invention,
iii) contacting said immunoglobulin with said Fc gamma receptor, and
iv) determining the binding of said immunoglobulin to said Fc gamma receptor.

The immunoglobulin to be analyzed can be e.g. an isolated immunoglobulin, a mixture of immunoglobulins, or a sample.

Another aspect is a method for the determination of the binding of an immunoglobulin to a composition according to the invention, comprising the following steps

i) providing an immunoglobulin to be analyzed,
ii) providing a composition of recombinant human Fc gamma receptor according to the invention,
iii) contacting said immunoglobulin with said composition of recombinant human Fc gamma receptor, and
iv) determining the binding of said immunoglobulin to said composition of recombinant human Fc gamma receptor.

A “sample” according to the present invention may be any tissue or liquid sample. Preferably the sample will be a liquid sample like saliva, urine, whole blood, plasma or serum. Preferably the sample will be whole blood, plasma or serum. Preferably the sample is a cell-free sample, i.e. a sample containing no cells.

Conditions which are appropriate for binding of an immunoglobulin to a receptor according to the invention are well-known to a person skilled in the art or can easily be determined. Under these conditions the immunoglobulin binds to the receptor and an immunological complex between the immunoglobulin and the receptor is formed, resulting in an immunoglobulin-receptor-complex. This complex can be detected by any appropriate means.

In one embodiment the immunoglobulin-receptor-complex is detected with the aid of an immunoassay. The immunoassay used preferably is a heterogeneous immunoassay. In one embodiment the detection of the immunoglobulin-receptor-complex is accomplished with a competitive immunoassay, or with a so-called sandwich immunoassay.

The skilled artisan will have no problem in setting up an immunoassay, which is capable of detecting the immunoglobulin-receptor-complex. By way of example such detection may be performed in a sandwich type immunoassay wherein an antibody is used as a capture antibody, which is binding to the immunoglobulin at an epitope which does not overlap with the epitope which is binding to the receptor. For detection of the immunoglobulin-receptor-complex it is possible to use a second or detection antibody to the receptor which binds to an epitope neither recognized by immunoglobulin nor by the capture antibody.

In one embodiment a detection antibody capable of forming a detection antibody-immunoglobulin-receptor-complex sandwich is used. Said second or detection antibody preferably is labeled in such a manner that direct or indirect detection is facilitated.

For direct detection the labeling group can be selected from any known detectable marker groups, such as dyes, luminescent labeling groups, such as chemoluminescent groups, e.g., acridinium esters or dioxetanes, or fluorescent dyes, e.g., fluorescein, coumarin, rhodamine, oxazine, resorufin, cyanine and derivatives thereof. Other examples of labeling groups are luminescent metal complexes, such as ruthenium or europium complexes, enzymes, e.g., as used for ELISA or for CEDIA (Cloned Enzyme Donor Immunoassay, e.g., EP 0 061 888), and radioisotopes.

Indirect detection systems comprise, for example, a detection reagent, e.g. the detection antibody, labeled with a first partner of a bioaffine binding pair. Examples of suitable binding pairs are hapten or antigen/antibody, biotin or biotin analogues such as aminobiotin, iminobiotin or desthiobiotin/avidin or Streptavidin, sugar/lectin, nucleic acid or nucleic acid analogue/complementary nucleic acid, and receptor/ligand, e.g., steroid hormone receptor/steroid hormone. Preferred first binding pair members comprise hapten, antigen and hormone. Especially preferred are haptens like digoxin, digoxigenin and biotin and analogues thereof. The second partner of such binding pair, e.g. an antibody, Streptavidin, etc., usually is labeled to allow for direct detection, e.g., by the labels as mentioned above.

Immunoassays are well known to the skilled artisan. Methods for carrying out such assays as well as practical applications and procedures are summarized in related textbooks. Examples of related textbooks are Tijssen, P., Preparation of enzyme-antibody or other enzyme-macromolecule conjugates, in: Practice and theory of enzyme immunoassays, Burdon, R. H. and v. Knippenberg, P. H. (eds.), Elsevier, Amsterdam (1990) pp. 221-278; and various volumes of Methods in Enzymology, Colowick, S. P., Caplan, N. O., Eds. “Methods in Enzymology”, dealing with immunological detection methods, especially volumes 70, 73, 74, 84, 92 and 121, Academic Press.

In all the above immunological detection methods conditions are chosen which allow for binding of the reagents employed, e.g. for binding of an immunoglobulin to its corresponding receptor. The immunoglobulin-receptor-complex detected according to the present invention is correlated by state of the art procedures to the corresponding concentration of the immunoglobulin or receptor in e.g. the sample.

In one embodiment is the Fc gamma receptor in the method according to the invention conjugated to a solid phase. In another embodiment is the immunoglobulin in the method according to the invention conjugated to a solid phase.

In a further embodiment is the conjugation of the Fc gamma receptor or the immunoglobulin performed by chemically binding via N-terminal and/or ε-amino groups (lysine), ε-amino groups of different lysines, carboxy-, sulfhydryl-, hydroxyl- and/or phenolic functional groups of the amino acid backbone and/or sugar alcohol groups of the carbohydrate structure. In another embodiment is the conjugation performed by passive adsorption. In still another embodiment is the conjugation of the Fc gamma receptor or the immunoglobulin performed via a specific binding pair selected from the specific binding pairs (first component/second component):

Streptavidin or Avidin/biotin,

antibody/antigen,

lectin/polysaccharide,

steroid/steroid binding protein,

hormone/hormone receptor,

enzyme/substrate, or immunoglobulin G/Protein A and/or G and/or L.

A further embodiment is that the determination of the binding of the immunoglobulin to the Fc gamma receptor is achieved by surface plasmon resonance, acoustic resonance, fluorescence resonance energy transfer, immunoassays, total internal reflection, fiber optics, surface plasmon resonance enhanced fluorescence, or fluorescence activated cell sorting. Preferred methods are surface plasmon resonance, enzyme linked immunoadsorbent assay, or fluorescence resonance energy transfer.

A “solid phase” denotes a non-fluid substance, and includes particles (including microparticles and beads) made from materials such as polymer, metal (paramagnetic, ferromagnetic particles), glass, and ceramic; gel substances such as silica, alumina, and polymer gels; capillaries, which may be made of polymer, metal, glass, and/or ceramic; zeolites and other porous substances; electrodes; microtiter plates; solid strips; and cuvettes, tubes or other spectrometer sample containers. A solid phase component of an assay is distinguished from inert solid surfaces with which the assay may be in contact in that a “solid phase” contains at least one moiety on its surface, which is intended to interact chemically with a molecule. A solid phase may be a stationary component, such as a chip, tube, strip, cuvette, or microtiter plate, or may be a non-stationary component, such as beads and microparticles. Microparticles can also be used as a solid phase for homogeneous assay formats. A variety of microparticles that allow both non-covalent or covalent attachment of proteins and other substances may be used. Such particles include polymer particles such as polystyrene and poly(methylmethacrylate); gold particles such as gold nanoparticles and gold colloids; and ceramic particles such as silica, glass, and metal oxide particles. See for example Martin, C. R., et al., Analytical Chemistry-News & Features, May 1 (1998) 322A-327A, which is incorporated herein by reference. The solid phase may optionally be coated, entirely or in certain areas. On the surface of the material any array of spots or an area is present, either visible or in coordinates. On each spot or the area, respectively, a polypeptide, with or without linker or spacer to the surface of the material, may be immobilized. Preferably the immobilized polypeptide is a receptor according to the current invention capable of binding the Fc part of an immunoglobulin of class G (IgG). Solid phases for immunoassays according to the invention are widely described in the state of the art (see, e.g., Butler, J. E., Methods 22 (2000) 4-23).

A solid-phase immunoassay with a receptor according to the invention, for example, involves the formation of a complex between an antibody adsorbed on or bound to a solid phase (capture antibody), the receptor, an immunoglobulin binding to the receptor, and an antibody binding to another epitope of the immunoglobulin or the receptor, which is conjugated to a detectable label (tracer antibody). Thus, a complex sandwich is formed: solid support-capture antibody-receptor-immunoglobulin-tracer antibody or support-capture antibody-immunoglobulin-receptor-tracer antibody. In the sandwich, the intensity of the tracer antibody-conjugated detectable label is proportional to the immunoglobulin concentration in the incubation medium. Mire-Sluis, A. R., et al., in J. Immunol. Methods 289 (2004) 1-16, summarize the recommendations for the design and optimization of immunoassays using detection of host antibodies against biotechnology products.

In an embodiment of the invention, either the receptor or the immunoglobulin is conjugated to a detectable label, preferably conjugated via a specific binding pair. Such a binding pair (first component/second component) is, for example, Streptavidin or Avidin/biotin, antibody/antigen (see, for example, Hermanson, G. T., et al., Bioconjugate Techniques, Academic Press, 1996), lectin/polysaccharide, steroid/steroid binding protein, hormone/hormone receptor, enzyme/substrate, IgG/Protein A and/or G and/or L, etc. Preferably, the conjugation is via digoxigenin and an antibody against digoxigenin to the detectable label. Alternatively the receptor or the immunoglobulin is conjugated to an electrochemiluminescent label, like a ruthenium bispyridyl complex.

The principles of different immunoassays are described, for example, by Hage, D. S., in Anal. Chem. 71 (1999) 294R-304R. Lu, B., et al., in Analyst 121 (1996) 29R-32R, report the orientated immobilization of antibodies for the use in immunoassays. Avidin-biotin-mediated immunoassays are reported, for example, by Wilchek, M. and Bayer, E. A., in Methods Enzymol. 184 (1990) 467-469.

Antibodies, especially their constant domains, contain amino acid side chain functionalities, i.e. chemical reactive groups, for coupling to a binding partner like a surface, a protein, a polymer (such as PEG, Cellulose, or Polystyrol), an enzyme, or a member of a binding pair. Chemical reactive groups of antibodies are, for example, amino groups (epsilon amino groups of lysines, alpha-amino groups), thiol groups (cystines, cysteines, and methionines), carboxylic acid groups (aspartic acids, glutamic acids), and sugar-alcoholic groups. Such methods are e.g. described by Aslam, M. and Dent, A., Bioconjuation MacMillan Ref. Ltd. (1999) 50-100.

For conjugation of polypeptides, e.g. to solid phases, suitable chemical protecting agents are required. These form e.g. bonds at unprotected side chain amines and are less stable than and different from those bonds at the N-terminus. Many such chemical protecting agents are known (see for example European Patent Application EP 0 651 761). Preferred chemical protecting agents include cyclic dicarboxylic acid anhydrides like maleic or citraconylic anhydrides.

Chromogens (fluorescent or luminescent groups and dyes), enzymes, NMR-active groups or metal particles, haptens, such as e.g. digoxigenin, are examples of “detectable labels”. The detectable label can also be a photoactivatable crosslinking group, e.g. an azido or an azirine group. Metal chelates which can be detected by electrochemoluminescence are also preferred signal-emitting groups, with particular preference being given to ruthenium chelates, e.g. a ruthenium (bispyridyl)₃²⁺ chelate. Suitable ruthenium labeling groups are described, for example, in EP 0 580 979, WO 90/05301, WO 90/11511, and WO 92/14138.

For the detection of immunoglobulin-receptor-complexes different methods can be employed, such as radioimmunoassay (RIA), enzyme linked immunosorbent assay (ELISA), immunoradiometric assays (IRMA), or surface plasmon resonance (SPR). In one embodiment the detection is by SPR, ELISA, or FRET (fluorescence resonance energy transfer)

The binding properties of an antibody, especially the KDiss., preferably is assessed by a BIAcore® instrument. In this method binding properties are evaluated by changes in surface plasmon resonance (SPR). It is convenient to bind the substance under investigation to the solid phase (called chip) and to assess binding e.g. of a monoclonal antibody, a polyclonal antibody, or even of serum comprising IgG to this coated chip. Such assay may be performed without washing steps (homogeneous immunoassay) or with washing steps (heterogeneous immunoassay).

In all the above immunological detection methods reagent conditions are chosen which allow for binding of the reagents employed, e.g. for binding of an antibody to its corresponding antigen. The skilled artisan refers to the result of such binding event by using the term complex. The complex formed in an assay method according to the present invention is correlated by state of the art procedures to the corresponding concentration of said antibody/immunoglobulin in the sample. Depending on the detection reagent employed this correlating step will result in the concentration of total, active or antigen-bound therapeutic antibody.

In order to provide in vitro data useful for the assessment of in vivo effects of, e.g., an immunoglobulin, an assay system has to be employed which resembles the in vivo conditions as precise as possible. First of all, assay compounds have to be used, which are at best identical to those in vivo. Today the compounds used in assay systems are recombinantly produced by biotechnological methods. For polypeptides employed in such assay systems it is among other things important to have the same amino acid sequence and glycosylation as the in vivo counterpart. Therefore, it is desirable to have production methods at hand, which allow for the production of polypeptides useful in assay systems whereby said polypeptides have the same amino acid sequence and glycosylation pattern as those produced in living mammals. The ratio behind this demand is that with assay systems resembling the in vivo conditions as close as possible more precise correlations of the assay results to in vivo effects can be obtained.

Therefore, the current invention comprises a method for the recombinant production of human Fc gamma receptor IIIa comprising the following steps:

providing a eukaryotic cell,

transfecting said provided eukaryotic cell with a heterologous nucleic acid encoding human Fc gamma receptor IIIa,

cultivating said transfected eukaryotic cell under conditions suitable for the expression of said human Fc gamma receptor IIIa,

recovering said recombinant human Fc gamma receptor IIIa from the eukaryotic cell or the cultivation medium,

whereby said recombinant human Fc gamma receptor IIIa is obtained as a mixture of at least two recombinant human Fc gamma receptor IIIa of SEQ ID NO: 1 differing in the N-linked oligosaccharide at amino acid position 163 of SEQ ID NO: 1.

In one embodiment is said eukaryotic cell a HEK 293 cell and said different oligosaccharides are selected from:

none, or

### HexNAc(β1→4) [Fuc(α1→3(6))]GlcNAc(β1→2)Man(α1→6(3)) [HexNAc(β1→4)GlcNAc(β1→2)Man(α1→3(6))]Man(β1→4)GlcNAc(β1→4) [Fuc(α1→6)]GlcNAcβ1-, or

### HexNAc(β1→4) [Fuc(α1→3(6))]GlcNAc(β1→2)Man(α1→6(3)) [Man(α1→6)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3)) [HexNAc(β1→4)[Fuc(α1→3(6))]GlcNAc(β1→2)Man(α1→3(6))]Man(β1→4)GlcNAc(β1→4)[Fuc(α1→6)]GlcNAcβ1-, or

### HexNAc(β1→4) [Fuc(α1→3(6))]GlcNAc(β1→2)Man(α1→6(3))[Man(α1→6)Man(α1→6)Man(α1→3(6))]Man(β1→4)GlcNAc(β1→4) [Fuc(α1→6)]GlcNAcβ1-, or

### HexNAc(β1→4) [Fuc(α1→3(6))]GlcNAc(β1→2)Man(α1→6(3))[Man(α1→6(3))Man(α1→3(6))]Man(α1→6)GlcNAc(β1→4) [Fuc(α1→6)]GlcNAcβ1-+HexNAc, or

### HexNAc(β1→4)[Fuc(α1→3(6))]GlcNAc(β1→2)Man(α1→6(3))[NeuAc(α2→6(3))Gal(β1→4)GlcNAc(β1→2)Man(α1→3(6))]Man(β1→4)GlcNAc(β1→4)[Fuc(α1→6)]GlcNAcβ1-, or

Gal(β1→4) [Fuc(α1→3(6))]GlcNAc(β1→2)Man(α1→6) [Gal(α1→4)[Fuc(α1→3(6))]GlcNAc(α1→2)Man(α1→3)]Man(β1→4)GlcNAc(β1→4)[Fuc(α1→6)]GlcNAcβ1-, or Gal(β1→4) [Fuc(α1→3(6))]GlcNAc(β1→2)Man(α1→6 (3))[Fuc(α1→3(6))GlcNAc(β1→2)Man(α1→3(6))]Man(β1→4)GlcNAc(β1→4)[Fuc(α1→6)]GlcNAcβ1-, or

### HexNAc(β1→4)GlcNAc(β1→2)Man(α1→6)[HexNAc(β1→4)GlcNAc(β1→2)Man(α1→3)]Man(β1→4)GlcNAc(β1→4)[Fuc(α1→6)]GlcNAcβ1-.

In another embodiment is said eukaryotic cell a CHO cell and said different oligosaccharides are selected from:

none, or

### NeuAc(α2→6(3)Gal(β1→4)GlcNAc(β1→6)Man(α1→6) [Gal(β1→4)GlcNAc(β1→4)Man(α1→3)]Man(β1→4)GlcNAc(β1→4) [Fuc(α1→6)]GlcNAcβ1-, or

### NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4) [Fuc(α1→6)]GlcNAcβ1-.

The examples, sequence listing and figures contained herein are provided to aid the understanding of the present invention, the true scope of which is set forth in the appended claims. It is understood that modifications can be made in the procedures set forth without departing from the spirit of the invention.

### EXAMPLE 1

### Plasmid pCLF60

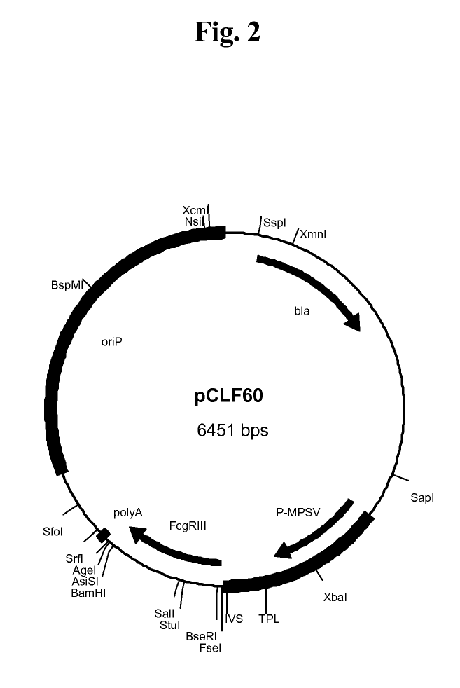

The plasmid pCLF60 has been constructed to express soluble human FcγRIIIa receptor under control of the myeloproliferative sarcoma virus (MPSV) promoter. The plasmid contains an adenoviral tripartite leader sequence (TPL) and a synthetic intron (IVS). For episomal replication in HEK293 EBNA cells the oriP element was inserted. An annotated plasmid map is given in FIG. 2. For purification purposes a hexahistidine tag has been cloned C-terminally to the Fc gamma RIIIa encoding nucleic acid.

The same plasmid has also been used for the expression of soluble human Fc gamma receptor IIIa in CHO cells. Therefore, the nucleic acid sequence encoding the C-terminal hexahistidine tag has been removed.

### EXAMPLE 2

### a) Cell Culture and Transfection of HEK 293 Cells with Plasmid pCLF60, Expression, Isolation and Purification of HEK-Expressed Fc Gamma RIIIa with Hexahistidine Tag

### Cell Culture

Human embryonic kidney cells HEK293 EBNA (Invitrogen, Switzerland) were adapted to serum-free growth in a Ca-reduced and fortified medium (Schumpp, B., and Schlaeger, E. J., J. Cell Sci. 97 (Pt 4, 1990) 639-47; Schlaeger, E. J., J. Immunol. Methods 194 (1996) 191-199). The cells were routinely grown in spinner flasks (Bellco, Inotech AG, Dotlikon, Switzerland) with shaking at 80-100 rpm. The large scale culture and transfection were performed either in a 5 liter stirred tank (Infors, Switzerland) or in a 24 liter airlift bioreactor (Chemap, MBR, Zutrich, Switzerland).

Plasmid preparations for pCLF60 were performed using a commercially available kit (Nucleobond Ax, Macherey-Nagel AG, Switzerland). The plasmid concentrations were determined spectrophotometrically and estimated by agarose gel electrophoresis with pUC18 DNA as standard (Pharmacia Biotech, Zutrich, Switzerland).

### Transfection Procedure

For transfection experiments, cells were cultured to a density of 6−10×10⁵ cells/ml, centrifuged for 5 minutes at 460×g (Heraeus-Kendro, Germany), washed once with heparin-free HL medium and resuspended in heparin-free HL medium (the calcium-free base HL medium is a mixture of enforced DHI and RPMI 1640 medium (2:1 wt/wt) as described by Schlaeger, E. J., J. Immunol. Methods, 194 (1996) 191-199). The cell concentration was adjusted to 5.5−6×10⁵ cells/ml and the culture was incubated in the bioreactor between 1-2 hours before the transfection took place. The transfection complexes were added aseptically to the bioreactor and cells were incubated for 4 days before harvesting the supernatant. Cells were fed with a concentrated feeding solution containing glucose, glutamine and peptones (Schumpp, B. and Schlaeger, E. J., J. Cell Sci. 97 (Pt 4, 1990) 639-647; Schlaeger, E. J., J. Immunol. Methods 194 (1996) 191-199).

### Preparation of Transfection Complexes

The DNA complexes were formed in 1/10 of the culture volume in HL medium without heparin at room temperature. Under optimized gene delivery conditions, 0.4 μg DNA was added for 1 ml HEK 293 EBNA cells to 0.1 ml fresh medium and mixed gently. After two minutes 1 μl Xtreme Gene (Roche Applied Science, Indianapolis, USA) was added and mixed. After incubating for 15 minutes at room temperature, the transfection complex was transferred to the equivalent amount of cells and cultured at 37° C. (Schlaeger, E. J. and Christensen, K., Cytotechnology 30 (1999) 71-83).

### Harvest and Purification

Supernatants were harvested by a depth filtration step (Cuno Filter Systems, Switzerland). Afterwards a concentration and buffer exchange step with an ultrafiltration unit (Millipore Helicon™ Filtration cartridge) using a 10 kD cut-off cellulose membrane (Millipore, Switzerland) was performed. The cell culture supernatant was exchanged with a 50 mM sodium phosphate buffer, pH 8.0, containing 500 mM NaCl and filter sterilized prior to purification. The ultrafiltrated solution was applied to a 0.22 μm filter prior to chromatographical purification. The obtained protein solution was supplemented with imidazole to a final concentration of 10 mM. This solution was applied to a Ni-NTA column at 4° C. with a flow rate of 3 ml/min. The bound protein was eluted after a wash step with an imidazole gradient in 50 mM sodium phosphate buffer (pH 8.0, supplemented with 500 mM NaCl) from 0 to 500 mM imidazole. The product containing fractions were identified by SDS-PAGE electrophoresis with Coomassie Brilliant Blue staining.

The product containing fractions were pooled and concentrated for a size exclusion chromatography (SEC) on a Sephacryl® S200 SEC column. The SEC chromatography was performed with a 25 mM sodium phosphate buffer, pH 7.4, supplemented with 100 mM sodium chloride.

### b) Transfection of CHO Cells with a Plasmid Encoding Fc Gamma RIIIa with an Avitag, Expression, Isolation and Purification of CHO-Expressed Fc gamma RIIIa with an Avitag

For the expression of the human Fc gamma RIIIa in CHO cells an expression plasmid based on pCLF60 has been used. In this plasmid the expression cassette for the Fc gamma RIIIa encodes an Avitag for post purification biotinylation.

For the preparation of CHO cells expressing human Fc gamma RIIIa, cultivation and isolation as well as purification an identical procedure as outlined above has been used.

After the chromatographical purification an enzymatic biotinylation with e.g. the enzyme biotin holoenzyme synthetase (BirA) of E. coli (a biotin ligase) can be performed.

### EXAMPLE 3

### Elucidation of the Glycosylation of the Expressed Fc Gamma RIIIa

The peptides obtained from a combined Endoproteinase Glu-C/Chymotrypsin digest (Roche Diagnostics GmbH, Germany) were separated using reversed phase liquid chromatography. The eluate was split for tandem mass spectrometry and parallel mass triggered fraction collection. The peptide digest was separated by reversed phase HPLC (Ultimate 3000, Dionex Corp., USA) equipped with an auto sampler, dual wavelength UV detector with a nano flow cell and temperature controlled column compartment (Ultimate 3000, Dionex Corp.). A Hypersil Gold C18 column, (250×0.3 mm I.D., 5 μm particle size, 175 Å pore size, Thermo Fisher Inc., USA) was used for separation. The solvents were A: 0.1% (v/v) formic acid (Sigma Aldrich) in water and B: 0.1% formic acid in acetonitrile (Baker). The column was equilibrated with 2 vol % B and the following gradient was applied using a flow rate of 5 μL/min: for 10 min. 2 vol % B, in 40 min. to 50 vol % B, in 40 min. to 80 vol % B, in 4 min. to 95 vol % B, for 2 min. 95 vol % B, for 25 min. 2 vol % B. The digested peptides were injected without pretreatment to the column.

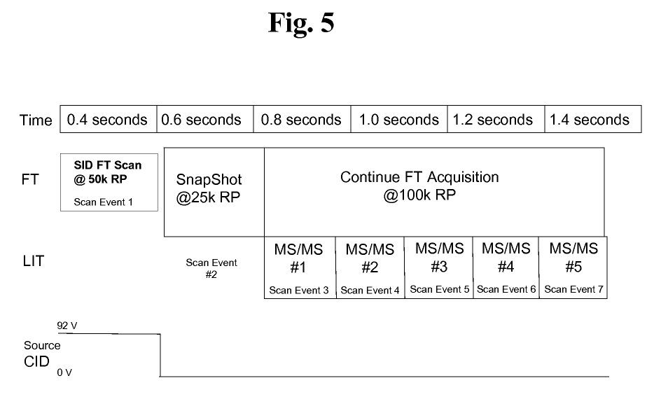

The eluate was split to a ratio of 1:20 using Triversa NanoMate (Advion) and approx. 200 mL/min were used for tandem mass spectrometry (LTQ FT ICR, Thermo Fisher Corp.). The remaining 4.8 μL/min were used for mass triggered fraction collection. A temporal representation of the scan event cycle employed for on-line identification and characterization of glycosylated and non-glycosylated enzymatic peptides using the FT ICR cell for accurate mass MS and the linear ion trap for high sensitivity MS/MS is shown in FIG. 5.

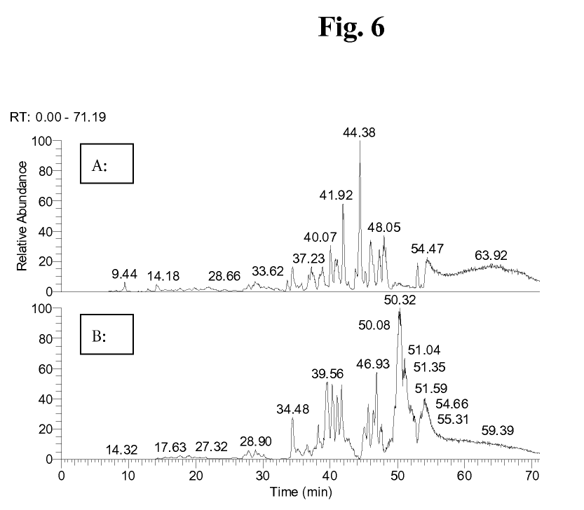

A typical total ion chromatogram for the reversed phase separation of an Endoproteinase Glu-C/Chymotryptic digest and the corresponding selected ion chromatogram for the performed SID-scan is shown in FIG. 6. Is clearly can be seen, the SID scan is very helpful in identifying the elution of glycopeptides.

Due to the heterogeneous glycosylation a number of different oligosaccharides are present at the different glycosylation sites. For example, for the position 163 of SEQ ID NO: 1 the following oligosaccharides can be found.

TABLE 1
Relative abundance of Asn 163-linked oligosaccharides in HEK 293
expressed recombinant human Fc gamma receptor IIIa.

| # | Mass | Relativeintensity(150 V): | 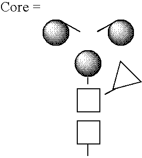 Glycosylation |
|---|---|---|---|
| 1 | 2431.0519 | 12.09 | Core + Fucose |
| 2 | 2634.1308 | 14.62 | Core + HexNAc + Fucose |
| 3 | 2837.2150 | 31.32 | Core + 2 HexNAc + Fucose |
| 4 | 2983.2739 | 17.05 | Core + 2 HexNAc + 2 Fucose |
| 5 | 2999.2655 | 17.72 | Core + 2 HexNAc + Hexose + Fucose |
| 6 | 3040.2919 | 14.37 | Core + 3 HexNAc + Fucose |
| 7 | 3145.3257 | 52.00 | Core + 2 HexNAc + Hexose + 2 Fucose |
| 8 | 3161.3197 | 20.66 | Core + 2 HexNAc + 2 Hexose + Fucose |
| 9 | 3186.3545 | 9.79 | Core + 3 HexNAc + 2 Fucose |
| 10 | 3202.3480 | 22.68 | Core + 3 HexNAc + Hexose + Fucose |
| 11 | 3243.3738 | 25.61 | Core + 4 HexNAc + Fucose |
| 12 | 3307.3814 | 30.21 | Core + 2 HexNAc + 2 Hexose + 2 Fucose |
| 13 | 3348.4159 | 36.19 | Core + 3 HexNAc + Hexose + 2 Fucose |
| 14 | 3389.4304 | 100.00 | Core + 4 HexNAc + 2 Fucose |
| 15 | 3535.4898 | 52.96 | Core + 4 HexNAc + 3 Fucose |

### EXAMPLE 4

### BIAcore-Assay

### a) Fc Gamma RIIIa Expressed in HEK 293 Cells

The isolated receptor of Example 2a) was amine coupled via a Lysine residue to the dextran matrix of a CM5 chip surface. For this the dextran matrix is firstly activated by a mixture of EDC (1-Ethyl-3-[3-dimethylaminopropyl]carbodiimide Hydrochloride) and NHS (N-hydroxy succinimide). The receptor-dilution is injected and amine coupled via amine groups.

The receptor was diluted to a concentration of 0.05 mg/ml with sodium acetate buffer or maleic acid buffer of pH 6.0-6.5. The coupling was performed with a flow rate set to 5 micro liters per minute. The injection time was set to 25 minutes. The 7 minutes activation of the dextran matrix by the EDC/NHS-mixture was followed by the 25 minutes injection (volume 125 micro liters) of the receptor dilution. A level of 893 RU has been immobilized.

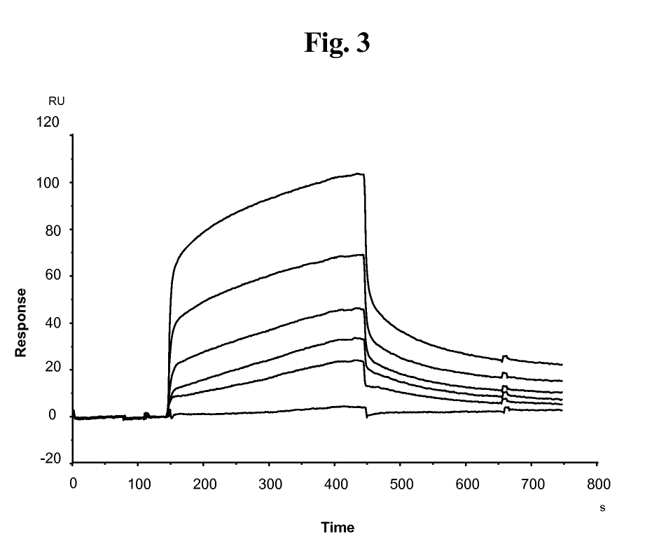

An anti-IGF-1R-antibody (e.g. as reported in WO2004/087756) was dissolved at different concentrations of from 6.25 to 100 nM in 50 mM HBS-P buffer (BIAcore; 0.01 M HEPES pH 7.4, 0.15 M NaCl, 0.005% Surfactant P-20). The solution of the antibody was contacted with the above prepared flow cell in a BIACORE®3000 instrument. Association with the immobilized receptor was measured by an injection of 5 minutes; dissociation was measured by washing the chip surface with antibody-free buffer for 5 minutes. A maximum response of 105 RU was recorded (FIG. 3).

### b) Fc Gamma RIIIa Expressed in CHO Cells

The isolated receptor of Example 2b) was immobilized on the surface of an avidin/streptavidin coated CM5 chip.

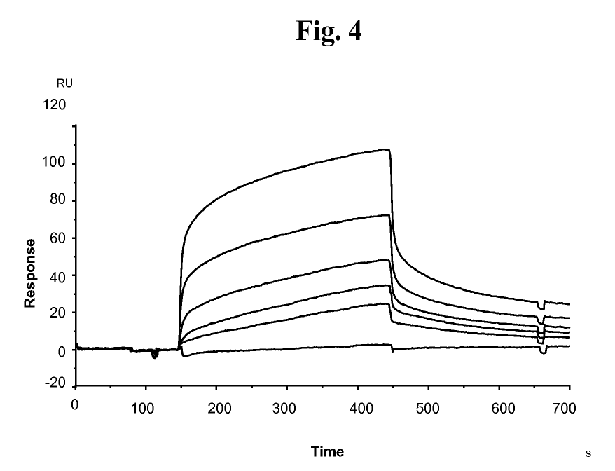

The receptor was diluted to a concentration of 0.05 mg/ml with sodium acetate buffer or maleic acid buffer of pH 6.0-6.5. The coupling was done with a flow rate set to 5 micro liters per minute. The injection time was set to 25 minutes. A level of 916 RU has been immobilized. An anti-IGF-1R-antibody was dissolved at different concentrations of from 6.25 to 100 nM in 50 mM HBS-P buffer (BIAcore; 0.01 M HEPES pH 7.4, 0.15 M NaCl, 0.005% Surfactant P-20). The solution of the antibody was contacted with the above prepared flow cell in a BIACORE®3000 instrument. Association with the immobilized receptor was measured by an injection of 5 minutes; dissociation was measured by washing the chip surface with antibody-free buffer for 5 minutes. A maximum response of 105 RU was recorded (FIG. 4).

1. A. composition of recombinant human Fc gamma receptor IIIa, wherein
a) said recombinant human Fc gamma receptor IIIa has the amino acid sequence of SEQ ID NO: 1 and has been expressed in HEK 293 cells, and
b) said composition comprises a mixture of at least two recombinant human Fc gamma receptor IIIa of SEQ ID NO: 1 differing in the N-linked oligosaccharide at amino acid position 163 of SEQ ID NO: 1,
whereby said N-linked oligosaccharides are selected from the group consisting of
none, or
HexNAc(β1→4)[Fuc(α1→3(6))]GlcNAc(β1→2)Man(α1→6(3))[HexNAc(β1→4)GlcNAc(β1→2)Man(α1→3)(6))]Man(β1→4)GlcNAc(β1→4)[Fuc(α1→6)]GlcNAcβ1-,
HexNAc(β1→4) [Fuc(α1→3(6))]GlcNAc(β1→2)Man(α1→6(3))[Man(α1→6)Man(α1→3(6))]Man(β1→4)GlcNAc(β1→4) [Fuc(α1→6)]GlcNAc 1-,
HexNAc(β1→4)[Fuc(α1→3(6))]GlcNAc(β1→2)Man(α1→6(3))[HexNAc(β1→4) [Fuc(α1→3(6))]GlcNAc(β1→2)Man(α1→3(6))]Man(β1→4)GlcNAc(β1→4) [Fuc(α1→6)]GlcNAcβ1-,
HexNAc(β1→4) [Fuc(α1→3(6))]GlcNAc(β1→2)Man(α1→6(3))[Man(α1→6)Man(α1→6)Man(α1→3(6))]Man(β1→4)GlcNAc(β1→4)[Fuc(α1→6)]GlcNAcβ1-,
HexNAc(β1→4) [Fuc(α1→3(6))]GlcNAc(β1→2)Man(α1→6(3))[Man(α1→6(3))Man(α1→3(6))]Man(β1→6)GlcNAc(β1→4) [Fuc(α1→6)]GlcNAcβ1-+HexNAc,
HexNAc(β1→4) [Fuc(α1→3(6))]GlcNAc(β1→2)Man(α1→6(3))[NeuAc(α2→6(3))Gal(β1→4)GlcNAc(β1→2)Man(α1→3(6))]Man(β1→4)GlcNAc(β1→4)[Fuc(α1→6)]GlcNAcβ1-,
Gal(β1→4) [Fuc(α1→3(6))]GlcNAc(β1→2)Man(α1→6) [Gal(α1→4)[Fuc(α1→3(6))]GlcNAc(α1→2)Man(α1→3)]Man(α1→4)GlcNAc(β1→4)[Fuc(α1→6)]GlcNAcβ1-,
Gal(β1→4) [Fuc(α1→3(6))]GlcNAc(β1→2)Man(α1→6(3)) [Fuc(α1→3(6))GlcNAc(β1→2)Man(α1→3(6))]Man(β1→4)GlcNAc(β1→4)[Fuc(α1→6)]GlcNAcβ1-, or
HexNAc(β1→4) GcNAc(β1→2)Man (α1→6) [HexNAc(β1→4)GlcNAc(β1→2)Man(α1→3)]Man(β1→4)GlcNAc(β1→4)[Fuc(α1→6)]GlcNAcβ1-.

2. A method for the determination of the binding of an immunoglobulin to the composition of claim 1, comprising the following steps
i) contacting an immunoglobulin with the composition of recombinant human Fc gamma receptor of claim 1, and,
ii) determining the binding of the immunoglobulin to said composition of recombinant human Fc gamma receptor.

3. The method of claim 2, wherein the recombinant human Fc gamma receptor or the immunoglobulin is conjugated to a solid phase.

4. The method of claim 3, wherein said conjugation is performed by chemically binding via N-terminal and/or ε-amino groups (lysine), ε-amino groups of different lysines, carboxy-, sulfhydryl-, hydroxyl- and/or phenolic functional groups of the amino acid backbone or sugar alcohol groups of the carbohydrate structure.

5. The method of claim 3, wherein said conjugation to the solid phase is performed via a specific binding pair selected from the group of specific binding pairs (first component/second component) consisting of:
Streptavidin or Avidin/biotin,
antibody/antigen,
lectin/polysaccharide,
steroid/steroid binding protein,
hormone/hormone receptor,
enzyme/substrate, and
immunoglobulin G/Protein A and/or G and/or L.

6. The method of claim 2, wherein said determination is by a method selected from the group consisting of surface plasmon resonance, acoustic resonance, fluorescence resonance energy transfer, immunoassay, total internal reflection, fiber optics, surface plasmon resonance enhanced fluorescence, and fluorescence activated cell sorting.

7. The method of claim 6, wherein said determination is by surface plasmon resonance.

8. The method according to claim 6, characterized in that said determination is by an immunoassay selected from the group consisting of a heterogeneous immunoassay and a sandwich assay.

9. The method of claim 8, wherein said determination is by sandwich assay, wherein said sandwich immunoassay comprises a capture antibody immobilized to a solid phase and a detection antibody suited for direct or indirect detection.

10. The method of claim 9, wherein said detection antibody comprises a detectable label selected from chemoluminescent groups, fluorescent groups, luminescent metal complexes, enzymes, and radioisotopes.

11. The method of claim 9, wherein said detection antibody comprises a first partner of a bioaffine binding pair selected from the group consisting of:
hapten or antigen/antibody,
biotin or biotin analogues such as aminobiotin, iminobiotin or desthiobiotin/avidin or Streptavidin,
sugar/lectin,
nucleic acid or nucleic acid analogue/complementary nucleic acid, and
receptor/ligand.

12. A method for the recombinant production of human Fc gamma receptor IIIa comprising:
transfecting a provided eukaryotic cell with a nucleic acid encoding human Fc gamma receptor IIIa,
cultivating said transfected eukaryotic cell under conditions suitable for the expression of said human Fc gamma receptor IIIa,
recovering said recombinant human Fc gamma receptor IIIa from the eukaryotic cell or the cultivation medium,
whereby the recovered recombinant human Fc gamma receptor IIIa is obtained as a composition comprising a mixture of at least two recombinant human Fc gamma receptor IIIa of SEQ ID NO: 1 differing in the N-linked oligosaccharide at amino acid position 163 of SEQ ID NO: 1.

13. The method of claim 12, wherein said eukaryotic cell is a HEK 293 cell and said oligosaccharides are selected from the group consisting of:
none,
HexNAc(β1→4) [Fuc(α1→3(6))]GlcNAc(β1→2)Man(α1→6(3))[HexNAc(β1→4)GlcNAc(β1→2)Man(α1→3(6))]Man(β1→4)GlcNAc(β1→4) [Fuc(α1→6)]GlcNAcβ1-,
HexNAc(β1→4) [Fuc(α1→3(6))]GlcNAc(β1→2)Man(α1→6(3))[Man(α1→6)Man(α1→3(6))]Man(β1→4)GlcNAc(β1→4) [Fuc(α1→6)]GlcNAcβ1-,
HexNAc(β1→4) [Fuc(α1→3(6))]GlcNAc(β1→2)Man(α1→6(3))[HexNAc(β1→4)[Fuc(α1→3(6))]GlcNAc(β1→2)Man(α1→3(6))]Man(β1→4)GlcNAc(β1→4) [Fuc(α1→6)]GlcNAcβ1-,
HexNAc(β1→4) [Fuc(α1→3(6))]GlcNAc(β1→2)Man(α1→6(3))[Man(α1→6)Man(α1→6)Man(α1→3(6))]Man(β1→4) GlcNAc(β1→4)[Fuc(α1→6)]GlcNAcβ1-,
HexNAc(β1→4) [Fuc(α1→3(6))]GlcNAc(β1→2)Man(α1→6(3))[Man(α1→6(3))Man(α1→3(6))]Man(β1→6)GlcNAc(β1→4) [Fuc(α1→6)]GlcNAcβ1-+HexNAc,
HexNAc(β1→4) [Fuc(α1→3(6))]GlcNAc(β1→2)Man(α1→6(3))
[NeuAc(α2→6(3))Gal(β1→4)GlcNAc(β1→2)Man(α1→3(6))]Man(β1→4)GlcNAc(β1→4) [Fuc(α1→6)]GlcNAcβ1-,
Gal(β1→4)[Fuc(α1→3(6))]GlcNAc(β1→2)Man(α1→6)[Gal(α1→4)[Fuc(α1→3(6))]GlcNAc(α1→2)Man(α1→3)]Man(α1→4)GlcNAc(α1→4)[Fuc(α1→6)]GlcNAcβ1-,
Gal(β1→4) [Fuc(α1→3(6))]GlcNAc(β1→2)Man(α1→6(3))[Fuc(α1→3(6))GlcNAc(β1→2)Man(α1→3(6))]Man(β1→4)GlcNAc(β1→4) [Fuc(α1→6)]GlcNAcβ1-,
HexNAc(β1→4) GlcNAc(β1→2) Man(α1→6) [HexNAc(β1→4)GlcNAc(β1→2)Man(α1→3)]Man(β1→4)GlcNAc(β1→4)[Fuc(α1→6)]GlcNAcβ1-.

14. The method according to claim 12, characterized in that said eukaryotic cell is a CHO cell and said oligosaccharide is selected from:
none,
NeuAc(α2→6(3)Gal(β1→4)GlcNAc(β1→6)Man(α1→6) [Gal(β1→4)GlcNAc(β1→4)Man(α1→3)]Man(β1→4)GlcNAc(β1→4) [Fuc(α1→6)]GlcNAc 1-,
NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4)[Fuc(α1→6)]GlcNAcβ1-.

15. A composition comprising recombinant human Fc gamma receptor IIIa, wherein
a) said recombinant human Fc gamma receptor IIIa has the amino acid sequence of SEQ ID NO: 1 and has been expressed in CHO cells, and
b) said composition comprises a mixture of at least two recombinant human Fc gamma receptor IIIa of SEQ ID NO: 1 differing in the N-linked oligosaccharide at amino acid position 163 of SEQ ID NO: 1,
whereby said N-linked oligosaccharides are selected from the group consisting of:
none, or
NeuAc(α2→6(3)Gal(β1→4)GlcNAc(β1→6)Man(α1→6)[Gal(β1→4)GlcNAc(β1→4)Man(β1→3)]Man(β1→4)GlcNAc(β1→4)[Fuc(α1→6)]GlcNAcβ1-, or
NeuAc(α2→6(3)Gal(β1→4)GlcNAc(β1→6)Man(α1→6) [NeuAc(α2→6(3)Gal(β1→4)GlcNAc(β1→4)Man(α1→3)]Man(β1→4)GlcNAc(β1→4)[Fuc(α1→6)]GlcNAcβ1-.

16. A method for the determination of the binding of an immunoglobulin to the composition of claim 15, comprising the following steps
i) contacting an immunoglobulin with the composition of recombinant human Fc gamma receptor of claim 15, and
ii) determining the binding of the immunoglobulin to said composition of recombinant human Fc gamma receptor.

17. The method of claim 16, wherein the recombinant human Fc gamma receptor or the immunoglobulin is conjugated to a solid phase.

18. The method of claim 17, wherein said conjugation is performed by chemically binding via N-terminal and/or ε-amino groups (lysine), ε-amino groups of different lysines, carboxy-, sulfhydryl-, hydroxyl- and/or phenolic functional groups of the amino acid backbone or sugar alcohol groups of the carbohydrate structure.

19. The method of claim 17, wherein said conjugation to the solid phase is performed via a specific binding pair selected from the group of specific binding pairs (first component/second component) consisting of:
Streptavidin or Avidin/biotin,
antibody/antigen,
lectin/polysaccharide,
steroid/steroid binding protein,
hormone/hormone receptor,
enzyme/substrate, and
immunoglobulin G/Protein A and/or G and/or L.

20. The method of claim 16, wherein said determination is by a method selected from the group consisting of surface plasmon resonance, acoustic resonance, fluorescence resonance energy transfer, immunoassay, total internal reflection, fiber optics, surface plasmon resonance enhanced fluorescence, and fluorescence activated cell sorting.

21. The method of claim 20, wherein said determination is by surface plasmon resonance.

22. The method of claim 20, wherein said determination is by an immunoassay, selected from the group consisting of a heterogeneous immunoassay and a sandwich immunoassay.

23. The method of claim 22, wherein said sandwich immunoassay comprises a capture antibody immobilized to a solid phase and a detection antibody suited for direct or indirect detection.

24. The method of claim 23, wherein said detection antibody comprises a detectable label selected from chemoluminescent groups, fluorescent groups, luminescent metal complexes, enzymes, and radioisotopes.

25. The method of claim 23, wherein said detection antibody comprises a first partner of a bioaffine binding pair selected from:
hapten or antigen/antibody,
biotin or biotin analogues such as aminobiotin, iminobiotin or desthiobiotin/avidin or Streptavidin,
sugar/lectin,
nucleic acid or nucleic acid analogue/complementary nucleic acid, and
receptor/ligand.

26. A recombinant human Fc gamma receptor IIIa, wherein said receptor
a) comprises the amino acid sequence of SEQ ID NO: 1, and
b) comprises at amino acid position 163 of SEQ ID NO: 1 one of the following N-linked oligosaccharides:
none, or
HexNAc(β1→4) [Fuc(α1→3(6))]GlcNAc(β1→2)Man(α1→6(3))[HexNAc(β1→4)GlcNAc(β1→2)Man(α1→3(6))]Man(β1→4)GlcNAc(β1→4)[Fuc(α1→6)]GlcNAcβ1-,
HexNAc(β1→4) [Fuc(α1→3(6))]GlcNAc(β1→2)Man(α1→6(3))[Man(α1→6)Man(α1→3(6))]Man(α1→4)GlcNAc(β1→4)[Fuc(α1→6)]GlcNAcβ1−,
HexNAc(114) [Fuc(α1→3(6))]GlcNAc(β1→2)Man(α1→6(3))[HexNAc(β1→4) [Fuc(α1→3(6))]GlcNAc(β1→2)Man(α1→3(6))]Man(β1→4)GlcNAc(β1→4) [Fuc(α1→6)]GlcNAcβ1-,
HexNAc(114) [Fuc(α1→3(6))]GlcNAc(β1→2)Man(α1→6(3))[Man(α1→6)Man(α1→6)Man(α1→3 (6))]Man(β1→4)GlcNAc(β1→4) [Fuc(α1→6)]GlcNAcβ1-,
HexNAc(β1→4) [Fuc(α1→3(6))]GlcNAc(β1→2)Man(α1→6(3))[Man(α1→6(3))Man(α1→3(6))]Man(β1→6)GlcNAc(β1→4)[Fuc(α1→6)]GlcNAcβ1-+HexNAc,
HexNAc(β1→4) [Fuc(α1→3(6))]GlcNAc(β1→2)Man(α1→6(3))[NeuAc(α2→6(3))Gal(β1→4)GlcNAc(β1→2)Man(α1→3(6))]Man(β1→4)GlcNAc(β1→4) [Fuc(α1→6)]GlcNAcβ1-,
Gal(β1→4) [Fuc(α1→3(6))]GlcNAc(β1→2)Man(α1→6) [Gal(α1→4)[Fuc(α1→3(6))]GlcNAc(β1→2)Man(α1→3)]Man(β1→4)GlcNAc(β1→4)[Fuc(α1→6)]GlcNAcβ1-,
Gal(β1→4)[Fuc(α1→3(6))]GlcNAc(β1→2)Man(α1→6(3))[Fuc(α1→3(6))GlcNAc(β1→2)Man(α1→3(6))]Man(β1→4)GlcNAc(β1→4) [Fuc(α1→6)]GlcNAcβ1-, or HexNAc(β1→4) GlcNAc(β1→2) Man(α1→6)[HexNAc(β1→4)GlcNAc(β1→2)Man(α1→3)]Man(β1→4)GlcNAc(β1→4)[Fuc(α1→6)]GlcNAcβ1-.

27. The Fc gamma receptor of claim 26, wherein said receptor comprises at amino acid position 75 of SEQ ID NO: 1 one of the following N-linked oligosaccharides:
none, or
NeuAc(α2→6(3)Gal(β1→4)GlcNAc(β1→6) [NeuAc(α2→6(3)Gal(β1→4)GlcNAc(β1→2)]Man(α1→6) [NeuAc(α2→6(3)Gal(β1→4) GlcNAc(β1→2)[NeuAc(α2→6(3)Gal(β1→4)GlcNAc(β1→4)]Man(α1→3)]Man(β1→4)GlcNAc(β1→4)[Fuc(α1→6)]GlcNAcβ1-,
NeuAc(α2→6(3)Gal(β1→4)GlcNAc(β1→6)[NeuAc(α2→6(3)Gal(β1→4)GlcNAc(β1→2)]Man(α1→6) [Gal(β1→4)GlcNAc(β1→2) [Gal(β1→4)GlcNAc(β1→4)]Man(α1→3)]Man(β1→4)GlcNAc(β1→4) [Fuc(α1→6)]GlcNAcβ1-, or
NeuAc(α2→6(3)Gal(β1→4)GlcNAc(β1→6) [NeuAc(α2→6(3)Gal(β1→4)GlcNAc(β1→2)]Man(α1→6) [NeuAc(α2→6(3)Gal(β1→4) GlcNAc(β1β2)[Gal(β1→4)GlcNAc(β1→4)]Man(α1→3)]Man(β1→4)GlcNAc(β1→4)[Fuc(α1→6)]GlcNAcβ1-.

28. The Fc gamma receptor of claim 26, wherein said receptor comprises at amino acid position 46 of SEQ ID NO: 1 one of the following N-linked oligosaccharides:
Gal(β1→4)GlcNAc(β1→2)Man(α1→6(3)) [GlcNAc(β1→2)Man(α1→3(6))]Man(β1→4)GlcNAc(β1→4)[Fuc(α1→6)]GlcNAcβ1-,
Man(α1→3(6))Man(α1→3(6)) [Man(α1→3(6))]Man(β1→4)GlcNAc(β1→4) [Fuc(α1→6)]GlcNAcβ1-,
Gal(β1→4)GlcNAc(β1→2)Man(α1→6) [Gal(β1→4)GlcNAc(β1→2)Man(α1→3)]Man(β1→4)GlcNAc(α1→4) [Fuc(α1→6)]GlcNAcβ1-,
GlcNAc(β1→2)Man(α1→6(3)) [Man(α1→3(6))]Man(β1→4)GlcNAc(α1→4)[Fuc(α1→6)]GlcNAcβ1-+Hex, or
Man(α1→3(6))Man(α1→3(6)) [Man(α1→3(6))]Man(β1→4)GlcNAc(β1→4)GlcNAcβ1-.

29. The Fc gamma receptor of claim 26, wherein said receptor comprises at amino acid position 170 of SEQ ID NO: 1 the following N-linked oligosaccharide:
HexNAc(β1→4)[Fuc(α1→3(6))]GlcNAc(β1→2)Man(α1→6(3))[Gal(β1→4)GlcNAc(β1→2)Man(α1→3(6))]Man(β1→4)GlcNAc(β1→4)[Fuc(α1→6)]GlcNAcβ1-.

30. The Fc gamma receptor of claim 26, wherein said receptor comprises at amino acid position 180 or 181 of SEQ ID NO: 1 the following O-linked oligosaccharide:
NeuAc(α2→6(3)Gal(β1→4)[NeuAc(α2→4(3)]HexNAc(β1→6)-.

31. The Fc gamma receptor of claim 26, wherein said receptor has a phenylalanine at amino acid position 159 of SEQ ID NO: 1.

32. A method for the determination of the binding of an immunoglobulin to the Fc gamma receptor of claim 26, characterized in that it comprises the following steps
i) contacting an immunoglobulin with the Fc gamma receptor, and
ii) determining the binding of said immunoglobulin to said Fc gamma receptor.

33. The method of claim 32, wherein the Fc gamma receptor or the immunoglobulin is conjugated to a solid phase.

34. The method of claim 33, wherein said conjugation is performed by chemically binding via N-terminal and/or ε-amino groups (lysine), ε-amino groups of different lysines, carboxy-, sulfhydryl-, hydroxyl- and/or phenolic functional groups of the amino acid backbone or sugar alcohol groups of the carbohydrate structure.

35. The method of claim 33, wherein said conjugation to the solid phase is performed via a specific binding pair selected from the group of specific binding pairs (first component/second component) consisting of:
Streptavidin or Avidin/biotin,
antibody/antigen,
lectin/polysaccharide,
steroid/steroid binding protein,
hormone/hormone receptor,
enzyme/substrate, and
immunoglobulin G/Protein A and/or G and/or L.

36. The method of claim 32, wherein said determination is by a method selected from the group of methods comprising surface plasmon resonance, acoustic resonance, fluorescence resonance energy transfer, immunoassay, total internal reflection, fiber optics, surface plasmon resonance enhanced fluorescence, and fluorescence activated cell sorting.
